(19) United States
(12) Patent Application Publication
(10) Pub. No.: US 2022/0323494 A1
(43) Pub. Date: Oct. 13, 2022

(54) Novel Control Switch

(71) Applicant: GlaxoSmithKline Intellectual Property Development Limited, Brentford, Middlesex (GB)

(72) Inventors: Lewis Lee BRAYSHAW, Stevenage (GB); Nicole CHRIST, Stevenage (GB); Carlos MARTINEZ FLEITES, Stevenage (GB)

(21) Appl. No.: 17/283,061

(22) PCT Filed: Oct. 14, 2019
(86) PCT No.: PCT/EP2019/077820

§ 371 Date: Apr. 6, 2021

(30) Foreign Application Priority Data
Oct. 16, 2018 (GB) 1816839.3

(51) Int. Cl. A61K 35/17; C07K 14/475; C07K 14/725; C12N 15/86

(52) U.S. Cl. CPC A61K 35/17; C07K 14/475; C07K 14/7051; C07K 2319/03; C07K 2317/622; C07K 2319/33; C12N 15/86

(57) ABSTRACT

The invention relates to a chimeric antigen receptor (CAR) suitable for the treatment of human subjects, comprising:
(i) a signalling chain comprising a transmembrane domain and a signalling domain;
(ii) a non-signalling chain comprising a target binding domain and a transmembrane domain;

wherein, one of said chains further comprises a HIV Integrase catalytic core domain (CCD) or a functional fragment or variant thereof, and the other chain further comprises a LEDGF/p75 integrase binding domain (IBD), or a functional fragment or variant thereof.

### FIELD OF THE INVENTION

The invention relates to a chimeric antigen receptor (CAR) which provides control over CAR signalling activity.

### BACKGROUND TO THE INVENTION

Chimeric antigen receptors (CARs) are artificial T-cell receptors that are at the forefront of modern personalised therapies (Lee et al. (2012) Clin. Cancer Res., 18(10): 2780-90). They are being developed to treat cancers in patients that are resistant to conventionally available therapies and use a patient's own immune cells to combat the disease. The immune cells are genetically engineered ex vivo to express a CAR specific to a tumour antigen, and the cells subsequently transferred back to the patient. CARs reside on the surface of T cells and consist of ecto- and endodomains which are separated by a transmembrane domain. The ectodomain harbours a target binding region (e.g. a single chain variable fragment) that is directed towards an antigen solely expressed on diseased cells. The endodomain (usually comprising CD3ζ-CD28 or CD3ζ-41BB) faces the cytosol and transmits an activation signal to the T cell after the antigen is bound to the target binding region on the surface of the cell.

The first generation CARs comprised target binding domains attached to a signalling domain derived from the cytoplasmic region of the CD3zeta or Fc receptor gamma chains. First generation CARs were shown to successfully redirect T cells to the selected target, however, they failed to provide prolonged expansion and antitumor activity of T-cells in vivo. Therefore, second and third generation CARs have focussed on enhancing modified T cell survival and increasing proliferation by including co-stimulatory molecules, such as CD28, OX-40 (CD134) and 4-1BB (CD137).

However, a safety concern of this promising therapy has arisen through potential cross-reactivity to vital organs (such as the lung). In clinical trials, both on-target as well as off-target off-tumour toxicities have been observed in patients treated with CAR-T cells and two fatalities have been reported with CAR studies (Morgan et al. (2010) Mol. Ther., 18(4): 843-51). These toxicities are difficult to predict in animal models, and in contrast to small molecules and biologics, CAR-T cells are living-drugs that have unique pharmacokinetic (PK) profiles. Therefore, safety switches are being developed to turn off or reduce CAR-T cell killing activity and enable more controlled and safer therapies.

One type of safety switch is a suicide switch, where CAR-T cells are further engineered to express “suicide genes” or “elimination genes”, which allows selective destruction of CAR-T cells upon administration of an external agent. For example, incorporating herpes simplex virus thymidine kinase (HSV-TK) means that administration of the prodrug ganciclovir results in cell death by incorporation of GCV-triphosphate into replicating DNA. Another method is the use of inducible caspase 9 (iCasp9), a chimeric protein that binds the small molecule AP1903, leading to caspase 9 dimerization and ultimately apoptosis of the CAR-T cell. However, the major disadvantage of a suicide switch is that it's irreversible and the therapy is destroyed. Other disadvantages are that the administration of the external agent may not act fast enough (switch elements are often immunogenic (e.g. HSV-TK)) or that the switch may not achieve 100% efficacy (either because the suicide agent is not homogenous or robust enough).

Another strategy to regulate the activity of CARS is by controlling the assembly (ON-type) or disassembly (OFF-type) of the endo- and ectodomains of the CAR in vivo. This is achieved by separating the signalling domain and target binding domain of the CAR into signalling and non-signalling chains, then modulating the signalling activity by adding or removing a small molecule which can act as an inducer or inhibitor for the dimerisation of the two different chains. Advantages of this type of switch strategy are that the switching is reversible and signalling can be modulated by changing the concentration of the small molecule. However, the pharmacological properties of small molecules already in use are not always optimal, with no guarantee that the receptor turn-off kinetics are directly controllable by the dosage of the small molecule. Being able to directly control the receptor turn-off kinetics would mean that the CAR activity could be fine-tuned to a level that effectively treats the disease/condition whilst reducing any side effects to a minimum.

One example of a reversible ON-type assembly switch is the rapamycin-FKPB12-mTOR complex in which rapamycin induces the dimerisation of the signalling and non-signalling chains of the CAR (Wu et al. (2015) Science, 350(6258): aab4077). In the rapamycin CAR, activation is mainly controlled through varying the concentration of a compound. Deactivation of the rapamycin CAR by rapamycin withdrawal, on the contrary, not only depends on the compound concentration, but is also influenced by the rate of the complex dissociation and compound clearance, which are parameters difficult to control by compound dosage. Another disadvantage of this strategy is that rapamycin must be continually administered throughout the treatment period.

WO2016/030691 describes a reversible OFF-type disassembly switch which uses the interaction between the Tet repressor (TetR) and TetR interacting protein (TiP) to control the activation of the CAR. This mechanism works in the opposite way to the ON-type assembly switch because the addition of a small molecule disrupts the dimerization of the TetR-TiP domains, thus deactivating the CAR by separating the constituent chains. This means that the disrupter molecule only needs to be administered when the CAR needs switching off or its activity downregulated. However, the WO2016/030691 uses tetracycline binding protein/peptides derived from bacteria which are potentially immunogenic in human subjects.

Therefore, there is still a need in the art to develop reversible OFF-type disassembly switches for controlling CAR T cell therapies in humans.

### SUMMARY OF THE INVENTION

According to a first aspect of the invention there is provided a chimeric antigen receptor (CAR) suitable for the treatment of human subjects, comprising:
(i) a signalling chain comprising a transmembrane domain and a signalling domain;
(ii) a non-signalling chain comprising a target binding domain and a transmembrane domain;
wherein, one of said chains further comprises an HIV Integrase catalytic core domain (CCD) or a functional fragment or variant thereof, and the other chain further comprises a LEDGF/p75 integrase binding domain (IBD), or a functional fragment or variant thereof.

According to another aspect of the invention, there is provided a polynucleotide encoding the signalling chain, a polynucleotide encoding the non-signalling chain and a polynucleotide encoding the signalling and non-signalling chains of the CAR as defined herein.

According to another aspect of the invention, there is provided an expression vector comprising a polynucleotide as defined herein.

According to another aspect of the invention, there is provided an immunomodulatory cell comprising the CAR as defined herein.

According to another aspect of the invention, there is provided an immunomodulatory cell as defined herein for use in therapy.

According to another aspect of the invention, there is provided a pharmaceutical composition comprising the immunomodulatory cells as defined herein.

According to another aspect of the invention, there is provided a method of treating and/or preventing a disease, which comprises administering the pharmaceutical composition as defined herein to a human subject.

According to another aspect of the invention, there is provided a method of making an immunomodulatory cell that expresses the CAR as defined herein, comprising:
(a) transducing or transfecting a polynucleotide or the expression vector as defined herein, into said immunomodulatory cell; and
(b) expressing said polynucleotide or said expression vector in the immunomodulatory cell.

According to another aspect of the invention, there is provided an immunomodulatory cell obtained by the method as defined herein.

According to another aspect of the invention, there is provided a method of controlling the activity of the CAR as defined herein in a human subject, which comprises administering to the human subject undergoing treatment with the CAR with an agent that inhibits the LEDGF/p75-HIV Integrase interaction.

### BRIEF DESCRIPTION OF THE FIGURES

FIG. 1. Schematic organisation of OFF-switch chimeric antigen receptor (constructs 1 to 5) that incorporate fragments of HIV Integrase and human LEDGF/p75 protein. Dimerisation of components such as the HIV Integrase domain or CD8α hinge is omitted from the representation for clarity. scFv is an anti-human BCMA single chain variable region fragment, CD8α is the hinge and transmembrane domain of human CD8α, p75 is a fragment comprising the IBD of human LEDGF/p75 protein, IN is a fragment comprising the CCD of HIV integrase, 41BB is the intracellular domain of human 4-1BB protein and CD3ζ is the signalling domain of human CD3ζ. Components are ensembled with linkers described in Example 1.

FIG. 2. Activation of NFAT promoter in Jurkat cells triggered by the interaction with BCMA antigen presenting cells ARH-77-10B5 (black bars). Jurkat cells were transfected with Constructs 1 to 5 as described in Example 1. The effect of addition of BI224436 on the NFAT activation of these constructs is represented by the grey bar.

FIG. 3. Activation of NFAT promoter in Jurkat cells triggered by the interaction with BCMA antigen presenting cells ARH-77-10B5 (black bars). Jurkat cells were transfected with Constructs 5 to 6 as described in Example 2. The difference between onstructs 5 and 6 is a point mutation on the HIV integrase domain described in Example 2. The effect of addition of small molecule BI224436 on the NFAT activation of these constructs is represented by the grey bar.

FIG. 4. (A) Schematic organisation of OFF-switch chimeric antigen receptor constructs 6 and 7 that incorporate the fragment of HIV Integrase with Phe1332Lys mutation and the fragment of human LEDGF/p75 protein. Dimerisation of components such as HIV Integrase or CD8α hinge is omitted from the representation for clarity. scFv is an anti-human BCMA single chain variable region fragment, CD8α is the hinge and transmembrane domain of human CD8α, p75 is a fragment of human LEDGF/p75 protein, IN is a fragment of HIV integrase, 41BB is the intracellular domain of human 4-1BB protein and CD3ζ is the signalling domain of human CD3ζ. Components are assembled with linkers described in Example 2 and Example 3. (B) Activation of NFAT promoter in Jurkat cells triggered by the interaction with BCMA antigen presenting cells ARH-77-10B5 (black bars). Jurkat cells were transfected with constructs 6 and 7 as described in Example 1. The difference between constructs 6 and 7 is the swap of the LEDGF/p75 chain fragment with the scFv element and the swap of the HIV integrase fragment with the of CD3ζ domain. The effect of addition of BI224436 on the NFAT activation of these constructs is represented by the grey bar.

FIG. 5. Expression levels of OFF-switch CARs in transduced Jurkat cells. Jurkat cells transduced with Construct 7 to 12 were analysed by flow cytometry 20 days after transduction to measure the percentage of cells positive for OFF-switch CAR expression, indicated by anti-BCMA scFv expression. For all constructs, the same MOI was used for Jurkat cell transduction.

FIG. 6. Effect of small molecule BI224436 on the cytokine release profile of Construct 9 and Construct 10 in primary T-cells (Example 6).

FIG. 7. Effect of titration of small molecule BI224436 on the release of TNFα, IFN-γ and IL-2 from primary T-cells stimulated with antigen. Titration was carried out with T-cells transduced with Construct 13 (Example 6).

FIG. 8. Effect of small molecule BI224436 on the cytotoxic effect of T-cells on target cells (Example 7).

FIG. 9. (A) Expression levels of the OFF-switch CAR and an equivalent conventional CAR in transduced T-cells. Primary T-cells cells transduced with Construct 14 and 10 were analysed by flow cytometry 12 days after transduction to measure the percentage of cells positive for CAR expression, indicated by anti-BCMA scFv expression (B) Effect of small molecule BI224436 on the cytotoxic effect of T-cells transduced with the OFF-switch CAR (Construct 10) and T-cells transduced with an equivalent conventional CAR (Construct 14) (Example 8).

### DETAILED DESCRIPTION OF THE INVENTION

The present invention is a fast-acting reversible OFF-switch that can directly be controlled through different concentrations of a small molecule compound. In case of an adverse event, this system would allow for a rapid inactivation or downregulation of CAR signalling, followed by elimination of CAR T-cells through long term systemic corticosteroids administration, immune suppression with cell-specific mAbs or lympho-depleting chemotherapy (e.g. cyclophosphamide) if required.

The present invention is a CAR which comprises two different proteins, the signalling and non-signalling chains, wherein signalling from the CAR only takes place if the signalling and non-signalling chains form a complex. If the complex is disrupted then signalling is also disrupted. The risk of unwanted side effects is reduced by using a small molecule known to disrupt CAR signalling, which is also known to be safe in humans. Furthermore, the protein components used have low immunogenicity potential and are well characterised with small molecular sizes and adequate N- and C-termini for optimal fusions with the other CAR components.

### Definitions

Unless defined otherwise, all technical and scientific terms used herein have the same meaning as is commonly understood by one of skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2^{nd} ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc. which are incorporated herein by reference in their entirety) and chemical methods. All patents and publications referred to herein are incorporated by reference in their entirety.

The term “comprising” encompasses “including” or “consisting” e.g. a composition “comprising” X may consist exclusively of X or may include something additional e.g. X+Y.

The term “about” as used herein when referring to a measurable value such as an amount, a temporal duration, and the like, is meant to encompass variations of ±20% or ±10%, including ±5%, ±1%, and ±0.1% from the specified value.

The term “chimeric antigen receptors” (“CARs”) as used herein, refers to engineered receptors which comprising a target binding domain (which is usually derived from a monoclonal antibody or fragment thereof), optionally a spacer region, a transmembrane region, and one or more intracellular effector domains. CARS have also been referred to as chimeric T cell receptors or chimeric immunoreceptors (CIRs). CARs are genetically introduced into hematopoietic cells, such as T cells, to redirect the cells' specificity for a desired cell-surface antigen.

References to “CAR signalling” refer to signalling through the signalling domain of the CAR which results in immunomodulatory cell activation (e.g. triggering target cell killing and T cell activation). In the system described herein, target binding by the non-signalling chain which is co-localized with the signalling chain results in productive CAR signalling through the signalling domain present in the signalling chain. If, however, an agent is present which causes the signalling chain and non-signalling chain to become delocalized, then target binding by the receptor component results in non-productive signalling because no signal is activated through the signalling domain.

The term “safety switch” or “control switch” refers to a biochemical mechanism that can be activated on demand in order to control a biological process which can cause harm. Safety switches can be used in CAR molecules so that they can be controlled externally (i.e. via administration from outside of the cell) in order to enhance the safety of the CAR therapy. For example, the signalling and non-signalling chains of the CAR can be split into separate components. The components contain binding domains which interact and bring the signalling and non-signalling chains together in order to activate signalling when the target antigen is bound. The advantage of this system is that the interaction between the binding domains can be controlled externally, e.g. by administration of an agent which either disrupts or brings the binding domains together.

The term “LEDGF/p75” refers to the human Lens Epithelium-Derived Growth Factor protein. Other synonyms for this protein exist, including: PC4 and SFRS1-interacting protein, CLL-associated antigen KW-7, Dense fine speckles 70 kDa protein (DFS 70), or Transcriptional coactivator p75/p52. The N-terminal domain of LEDGF/p75 binds chromosomal DNA whilst its C-terminal domain interacts with catalytic core domain (CCD) of HIV Integrase (UniProt: 075475) tethering the HIV intasome onto host cell chromatin.

The term “LEDGF/p75 C-terminal region” refers to the amino acid sequence of the LEDGF/p75 protein from amino acid residue 411 to the C-terminus of the protein.

The term “HIV Integrase” refers to Human Immunodeficiency Virus (HIV) Integrase (IN) which is an enzyme that enables the retroviral genetic material to be integrated into the DNA of the infected cell. It is a 32 kDa protein produced from the C-terminal portion of the HIV pol gene product (UniProt: P12497) and is an attractive target for new anti-HIV drugs. However, it is to be understood that the integrase domain (including any fragment or variant thereof) used in the CAR as described herein may originate from any retrovirus, including any variant or subtype of HIV, for example HIV-1 or HIV-2.

The term “domain” refers to a folded protein structure which retains its tertiary structure independent of the rest of the protein. Generally domains are responsible for discrete functional properties of proteins and in many cases may be added, removed or transferred to other proteins without loss of function of the remainder of the protein and/or of the domain.

The term “target binding domain” as used herein is defined as an oligo- or polypeptide that is capable of binding a specific target, such as an antigen or ligand. In particular, the target may be a cell surface molecule. For example, the target binding domain may be chosen to recognise a target that acts as a cell surface marker on pathogenic cells, including pathogenic human cells, associated with a particular disease state. The target binding domain may be, for example, any type of protein which binds to an antigen.

The term “spacer region” as used herein, refers to an oligo- or polypeptide that functions to link the transmembrane domain to the target binding domain. This region may also be referred to as a “hinge region” or “stalk region”. The size of the spacer can be varied depending on the position of the target epitope in order to maintain a set distance (e.g. 14 nm) upon CAR:target binding.

The term “transmembrane domain” as used herein refers to the part of the CAR molecule which traverses the cell membrane.

The term “signalling domain” (also referred to as the “intracellular effector domain”) as used herein refers to the domain in the CAR which is responsible for intracellular signalling following the binding of the target binding domain to the target. The signalling domain is responsible for the activation of at least one of the normal effector functions of the immune cell in which the CAR is expressed. For example, the effector function of a T cell can be a cytolytic activity or helper activity including the secretion of cytokines.

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanised, multispecific antibodies, including bispecific antibodies, and heteroconjugate antibodies; a single variable domain (e.g., VH, VHH, VL, domain antibody (dAb™)), antigen binding antibody fragments, Fab, F(ab′)₂, Fv, disulphide linked Fv, single chain Fv, disulphide-linked scFv, diabodies, TANDABS™, etc. and modified versions of any of the foregoing.

The term “single variable domain” refers to a folded polypeptide domain comprising sequences characteristic of antibody variable domains. It therefore includes complete antibody variable domains such as VH, VHH and VL and modified antibody variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A single variable domain is capable of binding an antigen or epitope independently of a different variable region or domain. A “domain antibody” or “dAb™” may be considered the same as a “single variable domain”. A single variable domain may be a human single variable domain, but also includes single variable domains from other species such as rodent (for example, as disclosed in WO 00/29004), nurse shark and Camelid VHH dAbs™. Camelid VHH are immunoglobulin single variable domain polypeptides that are derived from camelid species including bactrian and dromedary camels, llamas, vicugnas, alpacas and guanacos, which produce heavy chain antibodies naturally devoid of light chains. Such VHH domains may be humanised according to standard techniques available in the art, and such domains are considered to be “single variable domains”. As used herein VH includes camelid VHH domains.

“Affinity” is the strength of binding of one molecule, e.g. the target binding protein of the CAR of the invention, to another, e.g. its target antigen, at a single binding site. The binding affinity of the target binding protein to its target may be determined by equilibrium methods (e.g. enzyme-linked immunoabsorbent assay (ELISA) or radioimmunoassay (RIA)), or kinetics (e.g. BIACORE™ analysis).

Sequence identity as used herein is the degree of relatedness between two or more amino acid sequences, or two or more nucleic acid sequences, as determined by comparing the sequences. The comparison of sequences and determination of sequence identity may be accomplished using a mathematical algorithm; those skilled in the art will be aware of computer programs available to align two sequences and determine the percent identity between them. The skilled person will appreciate that different algorithms may yield slightly different results.

Thus the “percent identity” between a query nucleic acid sequence and a subject nucleic acid sequence is the “Identities” value, expressed as a percentage, that is calculated by the BLASTN algorithm when a subject nucleic acid sequence has 100% query coverage with a query nucleic acid sequence after a pair-wise BLASTN alignment is performed. Such pair-wise BLASTN alignments between a query nucleic acid sequence and a subject nucleic acid sequence are performed by using the default settings of the BLASTN algorithm available on the National Center for Biotechnology Institute's website with the filter for low complexity regions turned off. Importantly, a query nucleic acid sequence may be described by a nucleic acid sequence identified in one or more claims herein.

Similarly, the “percent identity” between a query amino acid sequence and a subject amino acid sequence is the “Identities” value, expressed as a percentage, that is calculated by the BLASTP algorithm when a subject amino acid sequence has 100% query coverage with a query amino acid sequence after a pair-wise BLASTP alignment is performed. Such pair-wise BLASTP alignments between a query amino acid sequence and a subject amino acid sequence are performed by using the default settings of the BLASTP algorithm available on the National Center for Biotechnology Institute's website with the filter for low complexity regions turned off. Importantly, a query amino acid sequence may be described by an amino acid sequence identified in one or more claims herein.

The query sequence may be 100% identical to the subject sequence, or it may include up to a certain integer number of amino acid or nucleotide alterations as compared to the subject sequence such that the % identity is less than 100%. For example, the query sequence is at least 50, 60, 70, 75, 80, 85, 90, 95, 96, 97, 98, or 99% identical to the subject sequence. Such alterations include at least one amino acid deletion, substitution (including conservative and non-conservative substitution), or insertion, and wherein said alterations may occur at the amino- or carboxy-terminal positions of the query sequence or anywhere between those terminal positions, interspersed either individually among the amino acids or nucleotides in the query sequence or in one or more contiguous groups within the query sequence.

The term “autologous” as used herein, refers to cells from the same human subject. The term “allogeneic” as used herein, refers to cells of the same species that differ genetically to the cell in comparison.

The terms “human subject” and “patient” are used herein interchangeably.

The term “pharmaceutical composition” refers to a composition formulated in pharmaceutically-acceptable or physiologically-acceptable solutions for administration to a cell or subject. The compositions of the invention may be administered in combination with other agents as well, provided that the additional agents do not adversely affect the ability of the composition to deliver the intended therapy.

The term “cancer” (sometimes also referred to as “neoplasia”) refers to a disease caused by an uncontrolled division of abnormal cells in a part of the body. The uncontrolled division can often result in a mass, commonly referred to as a “tumour” or “neoplasm”.

The term “tumour associated antigen” or “tumour antigen” as used herein, refers to an antigen expressed on a tumour cell. This antigen may be uniquely or differentially expressed on a tumour cell when compared to a normal, i.e. non-cancerous, cell.

The CAR described herein may also be used in methods of treatment of a subject in need thereof. Treatment can be therapeutic, prophylactic or preventative. Treatment encompasses alleviation, reduction, or prevention of at least one aspect or symptom of a disease and encompasses prevention or cure of the diseases described herein.

The CAR described herein may be used in an effective amount for therapeutic, prophylactic or preventative treatment. A therapeutically effective amount of the CAR described herein is an amount effective to ameliorate or reduce one or more symptoms of, or to prevent or cure, the disease.

### Chimeric Antigen Receptors

According to a first aspect of the invention there is provided a chimeric antigen receptor (CAR) suitable for the treatment of human subjects, comprising:
(i) a signalling chain comprising a transmembrane domain and a signalling domain;
(ii) a non-signalling chain comprising a target binding domain and a transmembrane domain;
wherein, one of said chains further comprises a HIV Integrase catalytic core domain (CCD) or a functional fragment or variant thereof, and the other chain further comprises a LEDGF/p75 integrase binding domain (IBD), or a functional fragment or variant thereof.

Binding of the HIV Integrase domain to the LEDGF/p75 domain causes heterodimerization and co-localization of the signalling and non-signalling chains. Thus, when the target binding domain of the non-signalling chain binds to the target and the HIV Integrase domain and LEDGF/p75 domainsare bound, there is signalling through the signalling chain.

The CAR described herein uses the HIV Integrase-LEDGF/p75 protein-protein interaction in a reversible OFF-switch mechanism. A complex forms between the catalytic core of a HIV Integrase homodimer and the 4-helix bundle domain of human transcription activation LRDGF/p75 (Cherepanov et al. (2005) PNAS, 102(48): 17308-13). This particular protein-protein interaction is the target of HIV Integrase allosteric inhibitor research that has produced potent, well characterised and bioavailable compounds (Tsiang et al. (2012) J. Biol. Chem., 287(25): 21189-203; Christ & Debyser, (2013) Virology, 435(1): 102-9). A potential advantage of the CAR described herein over other published examples, which use tetracycline binding protein/peptides derived from bacteria (e.g. WO 2016/030691), is that the CAR consists of protein domains already found to exist in human subjects and could potentially have a lower immunogenicity potential. Further, the components of the proposed system are small in size (˜150 residues HIV Integrase catalytic core domain (CCD) and ˜80 residues LEDGF/p75 integrase binding domain (IBD)) with the positions of the terminal amino acid residues suitable to connect to the rest of CAR components.

In addition to small molecules, the protein-protein interaction can also be modulated by short peptides (Hayouka et al. (2010) Biochem. Biophys. Res. Commun., 394(2): 260-265).

It will be understood that the CAR described herein requires the HIV Integrase and LEDGF/p75 domains to bind to each other. In one embodiment, the CAR comprises a HIV Integrase catalytic core domain (CCD) or a functional fragment or variant thereof and a LEDGF/p75 integrase binding domain (IBD), or a functional fragment or variant thereof. It will be understood that other parts of the HIV Integrase and LEDGF/p75 proteins can also be included, for example additional residues in the N-terminal or C-terminal domain.

In one embodiment, the LEDGF/p75 IBD comprises residues 347-426 of the wild-type protein (Uniprot 075475). In a further embodiment, the LEDGF/p75 IBD comprises SEQ ID NO: 1.

In one embodiment, the HIV Integrase CCD comprises residues 1203-1355 of the wild-type protein (Uniprot P12497). In a further embodiment, the HIV Integrase CCD comprises SEQ ID NO: 2.

References to a “functional fragment” refer to fragments of the full, wild-type amino acid sequences which still retain the binding function of the wild type protein from which they are derived (i.e. still enable the binding domains to interact). Fragments may suitably comprise at least 10 amino acids in length, for example 25, 50, 75, 80, 90, 100, 110, 120, 130, 140, 150, 160, 170, 180, 190, 200, 250 or 300 amino acids in length. Fragments may also comprise a C-terminal truncation, or an N-terminal truncation of the whole protein.

In one embodiment, the LEDGF/p75 functional fragment comprises residues 347-430 of the wild-type protein (Uniprot 075475). In a further embodiment, the LEDGF/p75 functional fragment comprises SEQ ID NO: 47.

In one embodiment, the LEDGF/p75 functional fragment comprises residues 347-442 of the wild-type protein (Uniprot 075475). In a further embodiment, the LEDGF/p75 functional fragment comprises SEQ ID NO: 48.

In one embodiment, the LEDGF/p75 functional fragment comprises residues 347-471 of the wild-type protein (Uniprot 075475). In a further embodiment, the LEDGF/p75 functional fragment comprises SEQ ID NO: 49.

In one embodiment, the HIV Integrase CCD comprises residues 1203-1355 of the wild-type protein (Uniprot P12497). In a further embodiment, the HIV Integrase CCD comprises SEQ ID NO: 2.

In one embodiment, the HIV Integrase functional fragment comprises residues 1203-1355 of the wild-type protein (Uniprot P12497). In a further embodiment, HIV Integrase comprises SEQ ID NO: 2.

References to a “functional variant” include variants with similar amino acid or nucleotide sequences to the wild-type sequences, but with one or more amino acid or nucleotide changes that result in a variant which still retains the binding function of the wild type protein from which they are derived (i.e. still enable the binding domains to interact). That is, provided that the functional variant facilitates sufficient co-localization of the receptor and intracellular signalling components for productive signalling to occur upon binding of the target to the target binding domain.

In one embodiment, the HIV Integrase functional variant comprises 80%, 85%, 90%, 95%, 98% or 99% sequence identity to the wild type sequence, provided that the sequence still enables binding to LEDGF/p75. Equally, in one embodiment, the LEDGF/p75 functional variant comprises 80%, 85%, 90%, 95%, 98% or 99% sequence identity to the wild type sequence, provided that the sequence still enables binding to HIV Integrase.

In one embodiment the LEDGF/p75 functional variant comprises one or more amino acid changes in the LEDGF/p75 C-terminal region, wherein amino acids with hydrophobic side chains are replaced with amino acids with hydrophilic or neutral side chains. Amino acids with hydrophobic side chains are Alanine, Valine, Isoleucine, Leucine, Methionine, Phenylalanine, Tyrosine and Tryptophan.

In one embodiment, the LEDGF/p75 functional variant comprises residues 347-430 of the wild-type protein (Uniprot 075475) with Leu428Gln and Val429Arg mutations. In a further embodiment, the HIV Integrase functional variant comprises SEQ ID NO: 50.

In one embodiment, the LEDGF/p75 functional variant comprises residues 347-430 of the wild-type protein (Uniprot 075475) with Leu428Gln and val429Gln mutations. In a further embodiment, the HIV Integrase functional variant comprises SEQ ID NO: 51.

In one embodiment, the HIV Integrase CCD comprises residues 1203-1355 of the wild-type protein (Uniprot P12497). In a further embodiment, the HIV Integrase CCD comprises SEQ ID NO: 2.

The binding of the signalling and non-signalling chains may be disrupted by the presence of a suitable agent. In a further embodiment, the invention further comprises an agent which disrupts the binding of the signalling and non-signalling chains. It will be understood that the term “agent” refers to any entity (e.g. a small drug molecule or peptide) that disrupts the interaction between HIV Integrase and LEDGF/p75 domains. This allows for the CAR signalling to be reversibly terminated in a controllable manner in order to avoid potential toxic side effects associated with continuous CAR signalling. The use of an agent also allows the potency of the CAR cells to be controlled pharmacologically and tuned to an acceptable balance between achieving the desired therapeutic effect and avoiding unwanted toxicities.

The disrupting agent may displace the signalling and non-signalling chains by preferentially binding to the signalling or non-signalling chain and thereby disrupting the heterodimerization required for signalling.

Use of the system described herein has the advantage that the therapy is not eliminated simply due to administration of the disrupting agent as is the case with suicide switches. The disrupting agent may also be administered to the patient before or simultaneously with the CAR in order to administer it in its “inactive” (i.e. OFF) state. Administering the CAR in its inactive state allows its distribution before activation. Once the CAR is activated, dosing with the disrupting agent is only necessary in case of severe side effects. If necessary, CAR-T cells can still be eliminated through methods known in the art, such as long term systemic corticosteroids administration, immune-suppression with cell-specific mAbs or lympho-depleting chemotherapy (e.g. cyclophosphamide).

The disrupting agent may be capable of being delivered to the cytoplasm of a target cell and available for intracellular binding. The disrupting agent may be capable of crossing the blood-brain barrier.

Agents known as “LEDGINs” have previously been described that act as potent inhibitors of the LEDGF/p75-HIV Integrase interaction by binding to the dimer interface of HIV Integrase (e.g. see Tsiang et al. (2012) J. Biol. Chem., 287(25): 21189-203; Christ & Debyser, (2013) Virology, 435(1): 102-9) and have been developed as antiviral agents for the treatment of HIV/AIDS. They can inhibit HIV replication with a dual mechanism of action: potent inhibition of the LEDGF/p75-HIV Integrase protein-protein interaction and allosteric inhibition of the catalytic function. Intensive drug discovery efforts over the past years have validated the LEDGF/p75-HIV Integrase interaction as a drugable target for antiviral therapy and has resulted in the design and synthesis of LEDGINs. Examples of agents which could be used in the CARs described herein have been optimised for clinical usage and are therefore suitable for human therapy. Therefore, in one embodiment, the disrupting agent is a LEDGIN.

In one embodiment, the disrupting agent is an inhibitor of the LEDGF/p75-HIV Integrase interaction. In a further embodiment, the disrupting agent is selected from a 2-(quinolin-3-yl)acetic acid derivative, a 2-(pyridine-3-yl)acetic acid derivative, a 2-(thieno[2,3-b]pyridine-5-yl)acetic acid derivative, or a (S)-2-(tert-butoxy)-2-phenylacetic acid derivative.

In one embodiment, the disrupting agent is a quinoline derivative, such as a 2-(quinolin-3-yl)acetic acid derivative. In a further embodiment, the disrupting agent is selected from: 3-Quinolineacetic acid, 4-(2,3-dihydropyrano[4,3,2-de]quinolin-7-yl)-α-(1,1-dimethylethoxy)-2-methyl-, (αS,4R)-; and 3-Quinolineacetic acid, 4-(3,4-dihydro-2H-1-benzopyran-6-yl)-α-(1,1-dimethylethoxy)-2-methyl-.

In one embodiment, the agent is selected from any of the agents described in Demeulemeester et al. (2014) Expert Opin. Ther. Pat. 24, 609-632, which is herein incorporated by reference.

In one embodiment, the agent is selected from any of the compounds listed in Table 1.

TABLE 1
Example compounds which inhibit LEDGF/p75-HIV Integrase interaction

| Structure | Name |
|---|---|
| 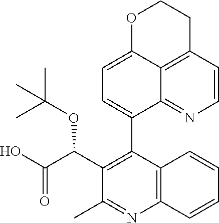 [SMILES: Cc1nc2ccccc2c(-c2ccc3c4c(ccnc24)CCO3)c1[C@@H](OC(C)(C)C)C(=O)O] | 3-Quinolineacetic acid, 4-(2,3- dihydropyrano[4,3,2-de]quinolin-7-yl)-α- (1,1-dimethylethoxy)-2-methyl-,(αS,4R)- (BI 224436) |
| 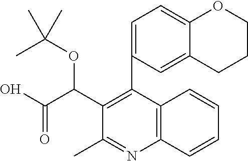 [SMILES: Cc1nc2ccccc2c(-c2ccc3c(c2)CCCO3)c1C(OC(C)(C)C)C(=O)O] | 3-Quinolineacetic acid, 4-(3,4-dihydro-2H-1- benzopyran-6-yl)-α-(1,1-dimethylethoxy)-2- methyl- |
| 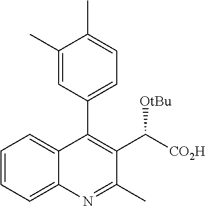 [SMILES: Cc1ccc(-c2c([C@H](OC(C)(C)C)C(=O)O)c(C)nc3ccccc23)cc1C] | (S)-2-(tert-butoxy)-2-(4-(3,4-dimethylphenyl)- 2-methylquinolin-3-yl)acetic acid |
| 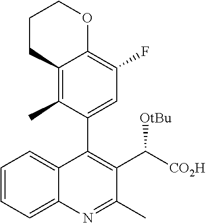 | (S)-2-(tert-butoxy)-2-((R)-4-(8-fluoro-5- methylchroman-6-yl)-2-methylquinolin-3- yl)acetic acid |
| 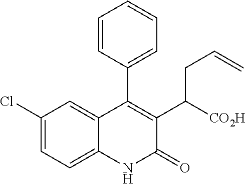 [SMILES: C=CCC(C(=O)O)c1c(-c2ccccc2)c2cc(Cl)ccc2[nH]c1=O] | 2-((6-chloro-2-oxo-4-phenyl-1,2- dihydroquinolin-3-yl)pent-4-enoic acid |
| 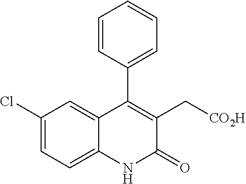 [SMILES: O=C(O)Cc1c(-c2ccccc2)c2cc(Cl)ccc2[nH]c1=O] | 2-((6-chloro-2-oxo-4-phenyl-1,2- dihydroquinolin-3-yl)acetic acid |
| 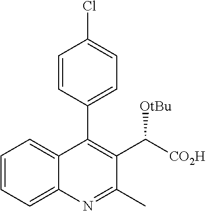 [SMILES: Cc1nc2ccccc2c(-c2ccc(Cl)cc2)c1[C@H](OC(C)(C)C)C(=O)O] | (S)-2-(tert-butoxy)-2-(4-(4-chlorophenyl)-2- methylquinolin-3-yl)acetic acid |
| 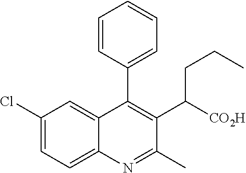 [SMILES: CCCC(C(=O)O)c1c(C)nc2ccc(Cl)cc2c1-c1ccccc1] | 2-(6-chloro-2-methyl-4-phenylquinolin-3- yl)pentanoic acid |
| 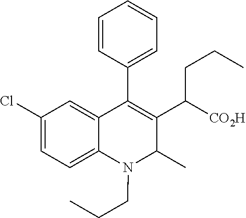 [SMILES: CCCC(C(=O)O)C1=C(c2ccccc2)c2cc(Cl)ccc2N(CCC)C1C] | 2-(6-chloro-2-oxo-4-phenyl-1-propyl-1,2- dihydroquinolin-3-yl)pentanoic acid |
| 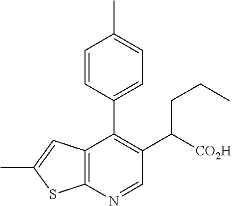 [SMILES: CCCC(C(=O)O)c1cnc2sc(C)cc2c1-c1ccc(C)cc1] | 2-(2-methyl-4-(p-tolyl)thieno[2,3-b]pyridin-5- yl)pentanoic acid |
| 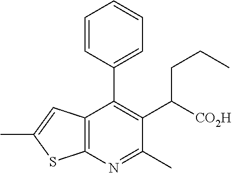 [SMILES: CCCC(C(=O)O)c1c(C)nc2sc(C)cc2c1-c1ccccc1] | 2-(2,6-dimethyl-4-phenylthieno[2,3-b]pyridin- 5-yl)pentanoic acid |
| 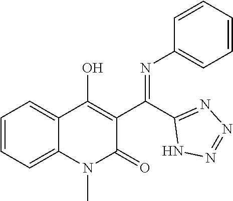 [SMILES: Cn1c(=O)c(/C(=N/c2ccccc2)c2nnn[nH]2)c(O)c2ccccc21] | (Z)-4-hydroxy-1-methyl-3-((phenylimino)(1H- tetrazol-5-yl)methyl)quinolin-2(1H)-one |
| 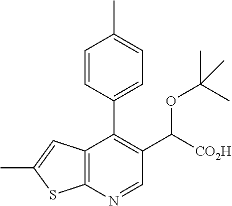 [SMILES: Cc1ccc(-c2c(C(OC(C)(C)C)C(=O)O)cnc3sc(C)cc23)cc1] | 2-(tert-butoxy)-2-(2-methyl-4-(p- tolyl)thieno[2,3-b]pyridin-5-yl)acetic acid |
| 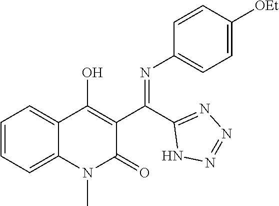 [SMILES: CCOc1ccc(/N=C(\c2nnn[nH]2)c2c(O)c3ccccc3n(C)c2=O)cc1] | (Z)-3-(((4-ethoxyphenyl)imino)(1H-tetrazol-5- yl)methyl)-4-hydroxy-1methylquinolin- 2(1H)-one |

In one embodiment, the signalling and non-signalling chains may comprise a peptide mimic of the HIV Integrase CCD or LEDGF/p75 IBD, which binds with lower affinity than the wild type HIV Integrase or LEDGF/p75 domains. This then allows the natural HIV Integrase or LEDGF/p75 domain to be used as the agent to disrupt the binding of a peptide mimic through competitive binding.

Antigen binding by the non-signalling chain in the absence of the disrupting agent may result in signalling through the signalling chain which is 2, 5, 10, 50, 100, 1000 or 10000-fold higher than the signalling which occurs when antigen is bound by the non-signalling chain in the presence of the disrupting agent.

The signalling and non-signalling chains may facilitate signalling through the CAR which is proportional to the concentration of the disrupting agent which is present. Thus, whilst the disrupting agent can displace the binding between the signalling and non-signalling chains, co-localization of the signalling and non-signalling chains may not be completely reduced in the presence of low concentrations of the disrupting agent. Therefore, low concentrations of the disrupting agent may decrease the level of signalling without completely inhibiting it. Levels of signalling and the correlation with concentration of the disrupting agent can be determined using methods known in the art.

CAR signalling may be determined by a variety of methods known in the art. For example, assays measuring signal transduction may be used, such as assaying levels of specific protein tyrosine kinases (PTKs), breakdown of phosphatidylinositol 4,5-bisphosphate (PIP₂), activation of protein kinase C (PKC) and elevation of intracellular calcium ion concentration. Functional readouts can also be used, such as measurement of clonal expansion of T cells, upregulation of activation markers on the cell surface, differentiation into effector cell and induction of cytotoxicity or cytokine (e.g. IL-2) secretion. For example, Bio-GIo™ NFAT luciferase Activation Assay from Promega is an example of a commercially available assay which can be used.

The present invention describes for the first time the use of an inhibitor of the LEDGF/p75-HIV Integrase interaction in a reversible CAR OFF-switch. Therefore, according to a further aspect of the invention, there is provided the use of the disrupting agents described herein for inhibiting a CAR as described herein.

A linker may be present between one or more of the domains that comprise the signalling and non-signalling chains. In one embodiment, the CAR additionally comprises a linker between the transmembrane domain and HIV Integrase or LEDGF/p75 domain, and/or between the transmembrane domain and the HIV Integrase or LEDGF/p75 domain, and/or between the signalling domain and the HIV Integrase or LEDGF/p75 domain. If a costimulatory domain is present, then the CAR may additionally comprise a linker between the costimulatory domain and an adjacent domain. For example, the linker may be between the transmembrane domain and the HIV Integrase or LEDGF/p75 domain, and/or between the HIV Integrase or LEDGF/p75 domain and the costimulatory domain, and/or between the transmembrane domain and the HIV Integrase or LEDGF/p75 domain, and/or between the HIV Integrase or LEDGF/p75 domain and the costimulatory domain, and/or between the signalling domain and the HIV Integrase or LEDGF/p75 domain, and/or between the signalling domain and the costimulatory domain.

Ideally, linkers connected to HIV Integrase are of sufficient length to enable dimerization with a HIV Integrase from a neighbouring component and orient it in the correct direction.

The linkers may be designed with sequences of adequate lengths that comply with the structural information reported in Cherepanov et al., (2005) PNAS, 102(48): 17308-13, with PDB accession code 2B4J. For example, the linker connecting a domain to the HIV Integrase or LEDGF/p75 domain, may be of sufficient length that allows for the rotation of the complex around the two axes that connect the N-termini of two copies of the LEDGF/p75 domain and the N-termini of two copies of HIV Integrase domain. Therefore, in one embodiment, the linker is at least 15 amino acid residues in length, such as 15, 16, 17, 18, 19, 20, 21, 22, 23, 24, 25, 26, 27, 28, 29, 30, 35, 40, 45, 50 or 55 amino acid residues in length. In a further embodiment, the linker between the transmembrane domain and LEDGF/p75 is at least 20 amino acid residues in length. In one embodiment, the linker between the transmembrane domain and HIV Integrase is at least 17 amino acid residues in length.

The linkers according to the invention may comprise alone, or in addition to other linkers, one or more sets of GS residues. In one embodiment, the linker comprises (GGGGS)ₙ(DPS)ₘ(GGS)ₚ, wherein n=1-10, m=0-3 and p=0-3. For example, in one embodiment, the linker comprises (GGGGS)ₙ(DPS)ₘ(GGS)ₚ, wherein n=4, m=1 and p=0. In an alternative embodiment, the linker comprises (GGGGS)ₙ(DPS)ₘ(GGS)ₚ, wherein n=4, m=0 and p=1.

In one embodiment, linker comprises (SDPS)_{q}(GGGGS)ₙ(GGS)ₚ, wherein n=1-10, p=0-3 and q=0-3. For example, in one embodiment, the linker comprises (SDPS)_{q}(GGGGS)n(GGS)ₚ, wherein n=3, p=0 and q=1. In an alternative embodiment, the linker comprises (SDPS)_{q}(GGGGS)ₙ(GGS)ₚ, wherein n=1, p=0 and q=1.

In one embodiment, the linker comprises at least 80%, 85%, 90%, 95%, 98% or 99% sequence identity to SEQ ID NOs: 3-9. In one embodiment, the linker comprises any one of SEQ ID NOs: 3-9 or a combination thereof.

In the linker sequences described herein, the addition of residues SPD at the beginning and/or end of the binding domains can be provided in order to break the N- and C-terminal helices of HIV Integrase CCD and/or LEDGF/p75 IBD.

It will be understood that different linkers can be used between different domains in the CAR described herein. Thus, in one embodiment, the linker between the transmembrane domain and the first binding domain and/or the transmembrane domain and the second binding domain, comprises SEQ ID NO: 3 or 4. In a further embodiment, the linker between the transmembrane domain and the first binding domain, comprises SEQ ID NO: 3 or 4. In a yet further embodiment, the linker between the transmembrane domain and LEDGF/p75, comprises SEQ ID NO: 3 or 4. It will be understood, that if costimulatory domains are included within the CAR, then the embodiments described herein may equally apply, for example, the linker between the costimulatory domain and LEDGF/p75, may comprise SEQ ID NO: 3 or 4.

In one embodiment, the linker between the signalling domain and the HIV Integrase or LEDGF/p75 domain, comprises any one of SEQ ID NOs: 5-7. In a further embodiment, the linker between the signalling domain and HIV Integrase domain, comprises any one of SEQ ID NOs: 5-7. In an alternative embodiment, the linker between the signalling domain and LEDGF/p75 domain, comprises any one of SEQ ID NOs: 5-7.

If a costimulatory domain is present, then the linker between the costimulatory domain and HIV Integrase or LEDGF/p75 domain, may comprise any one of SEQ ID NOs: 5-7. If the costimulatory domain is present after the HIV Integrase or LEDGF/p75 domain, then the linker sequence may comprise SEQ ID NO: 5 or 6, in particular SEQ ID NO: 5 for the LEDGF/p75 domain and/or SEQ ID NO: 6 for the HIV Integrase domain. In this embodiment, if the costimulatory domain is present between the HIV Integrase or LEDGF/p75 domain domain and the signalling domain, then the linker between the costimulatory domain and signalling domain may comprise SEQ ID NO: 7.

The target binding domain binds to a target, wherein the target is a tumour specific molecule, viral molecule, or any other molecule expressed on a target cell population that is suitable for mediating recognition and elimination by a lymphocyte. In one embodiment, the target binding domain comprises an antibody, an antigen binding fragment or a ligand. In one embodiment, the target binding domain comprises an antibody or fragment thereof. In one embodiment, the target binding domain is a ligand (e.g. a natural ligand of the target antigen). In an alternative embodiment, the target binding domain is an antigen binding fragment. In a further embodiment, the antigen binding fragment is a single chain variable fragment (scFv) or a dAb™. In a yet further embodiment, said scFv comprises the light (VL) and the heavy (VH) variable fragment of a target antigen specific monoclonal antibody joined by a flexible linker.

In one embodiment, the target binding domain may bind to more than one target, for example two different targets. Such a target binding domain may be derived from a bispecific single chain antibody. For example, Blinatumomab (also known as AMG 103 or MT103) is a recombinant CD19 and CD3 bispecific scFv antibody consisting of four immunoglobulin variable domains assembled into a single polypeptide chain. Two of the variable domains form the binding site for CD19 which is a cell surface antigen expressed on most normal and malignant B cells. The other two variable domains form the binding site for CD3 which is part of the T cell-receptor complex on T cells. These variable domains may be arranged in the CAR in tandem, i.e. two single chain antibody variable fragments (scFv) tethered to a spacer, and transmembrane and signalling domains. The four variable domains can be arranged in any particular order within the CAR molecule (e.g. VL(first target)-VH(first target)-VH(second target)-VL(second target) or VL(second target)-VH(second target)-VH(first target)-VL(first target) etc.).

The target binding domain may bind a variety of cell surface antigens, but in one embodiment, the target binding domain binds to a tumour associated antigen. In a further embodiment, the tumour associated antigen is selected from: BCMA, carcinoembryonic antigen (CEA), cancer antigen-125, CA19-9, CD5, CD13, CD19, CD20, CD22, CD27, CD30, CD33, CD34, CD45, CD52, CD70, CD117, CD138, CD160, epidermal growth factor receptor (EGFR), folate binding protein, ganglioside G2 (GD2), HER2, mesothelin, MUC-1, neural cell adhesion molecule (NCAM), prostate stem cell antigen (PSCA), prostate-specific membrane antigen (PSMA), prostatic acid phosphatise (PAP), protein melan-A, synaptophysis, six transmembrane epithelial antigen of the prostate I (STEAP1), TARP, Trp-p8, tyrosinase or vimentin. In a yet further embodiment, the tumour associated antigen is BCMA.

In one embodiment, the target binding domain has a binding affinity of less than about 500 nanomolar (nM), such as less than about 400 nM, 350 nM, 300 nM, 250 nM, 200 nM, 150 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 0.5 nM or 0.25 nM. In one embodiment, the target binding domain has a binding affinity of about 10 nM to about 0.25 nM. In a further embodiment, the target binding domain has a binding affinity of about 1 nM to about 0.5 nM (i.e. about 1000 μM to about 500 μM).

In one embodiment, the CAR additionally comprises a spacer domain between the target binding domain and the transmembrane domain. A spacer allows the target binding domain to orient in different directions to facilitate binding and can be used to improve the target binding interaction. In one embodiment, the spacer comprises a sequence derived from IgG (e.g. IgG1 Fc region or IgG1 hinge region), CD8 or CD4.

The transmembrane domain in each of the signalling and non-signalling chains acts as a membrane anchor to maintain the signalling chain at the cell surface.

In one embodiment, the transmembrane domain can be derived either from a natural or from a synthetic source. In one embodiment, the transmembrane domain can be derived from any membrane-bound or transmembrane protein. Alternatively the transmembrane domain can be synthetic and can comprise predominantly hydrophobic residues such as leucine and valine.

For example, the transmembrane domain can be the transmembrane domain of CD proteins, such as CD4, CD8, CD3 or CD28, a subunit of the T cell receptor, such as α, β, γ or δ, a subunit of the IL-2 receptor (α chain), or a subunit chain of Fc receptors. In one embodiment, the transmembrane domain comprises the transmembrane domain of CD4, CD8 or CD28. In a further embodiment, the transmembrane domain comprises the transmembrane domain of CD4 or CD8 (e.g. the CD8 alpha chain, as described in NCBI Reference Sequence: NP_001139345.1, incorporated herein by reference).

In one embodiment, the transmembrane domain comprises SEQ ID NO: 10.

In one embodiment the signalling chain may additionally comprise a hinge sequence next to the transmembrane domain. In a further embodiment, the hinge sequence comprises SEQ ID NO: 11. In a further embodiment, the hinge and transmembrane domain comprise the complete sequence of SEQ ID NO: 12.

In some embodiments, the transmembrane domain is composed of the CD8α transmembrane helix immediately followed by the full length intracellular domain of 4-1BB which contains a stretch of sequence compatible with the membrane interface. If the domain next to the transmembrane domain does not have a sequence compatible with the membrane interface then a linker may be used.

Thus, in one embodiment, there is a linker between the transmembrane domain and the domain immediately following the transmembrane domain on the intracellular side of the cell membrane. In a further embodiment, there is a linker between the transmembrane domain and the HIV Integrase or LEDGF/p75 domain; or, if present, the transmembrane domain and the costimulatory domain. In a yet further embodiment, the linker comprises SEQ ID NO: 13. This linker is especially advantageous if the transmembrane domain is derived from CD8α because it is simply the native sequence from CD8α that immediately follows the transmembrane helix.

Preferred examples of the signalling domain for use in a CAR described herein, can be the cytoplasmic sequences of the natural T cell receptor and co-receptors that act in concert to initiate signal transduction following antigen binding, as well as any derivate or variant of these sequences and any synthetic sequence that has the same functional capability. Signalling domains can be separated into two classes: those that initiate antigen-dependent primary activation, and those that act in an antigen-independent manner to provide a secondary or costimulatory signal. Primary activation effector domains can comprise signalling motifs which are known as immunoreceptor tyrosine-based activation motifs (ITAMs). ITAMs are well defined signalling motifs, commonly found in the intracytoplasmic tail of a variety of receptors, and serve as binding sites for syk/zap70 class tyrosine kinases. Examples of ITAMs used in the invention can include, as non-limiting examples, those derived from CD3zeta, FcRgamma, FcRbeta, FcRepsilon, CD3gamma, CD3delta, CD3epsilon, CD5, CD22, CD79a, CD79b and CD66d. In one embodiment, the signalling domain comprises a CD3zeta signalling domain (also known as CD247). In a further embodiment, the CD3zeta signalling domain comprises SEQ ID NO: 14. This sequence is also found in Uniprot P20963, residues 51-164. Natural TCRs contain a CD3zeta signalling molecule, therefore the use of this effector domain is closest to the TCR construct which occurs in nature.

In one embodiment, the signalling domain of the signalling chain comprises a CD3zeta signalling domain which has an amino acid sequence with at least 70%, preferably at least 80%, more preferably at least 85%, 90%, 95% 98% or 99% sequence identity with SEQ ID NO: 14. In a further embodiment, the signalling domain of the CAR comprises a CD3zeta signalling domain which comprises an amino acid sequence of SEQ ID NO: 14.

The signalling or non-signalling chain of the CAR may further comprise a signal peptide so that when the component is expressed in a cell, the nascent protein is directed to the endoplasmic reticulum and subsequently to the cell surface where it is expressed.

The core of the signal peptide may contain a long stretch of hydrophobic amino acids that has a tendency to form a single alpha helix. The signal peptide may begin with a short positively charged stretch of amino acids which helps to enforce proper topology of the polypeptide during translocation. At the end of the signal peptide there is typically a stretch of amino acids that is recognized and cleaved by signal peptidase. Signal peptidase may cleave either during or after completion of translocation to generate free signal peptide and a mature protein. The free signal peptides are then digested by specific proteases. The signal peptide may be at the amino terminus of the molecule.

In one embodiment, the signal peptide is derived from CD8 (see UniProt P01732). In a further embodiment, the signal peptide comprises SEQ ID NO: 15 or a variant thereof having 5, 4, 3, 2 or 1 amino acid mutations (insertions, deletions, substitutions or additions) provided that the signal peptide still functions to cause cell surface expression of the component (i.e. a functional variant).

As described herein, the signalling and/or non-signalling chain may further contain a secondary or costimulatory signal. T cells additionally comprise costimulatory molecules which bind to cognate costimulatory ligands on antigen presenting cells in order to enhance the T cell response, for example by increasing proliferation activation, differentiation and the like. Therefore, in one embodiment, the signalling and non-signalling chains additionally comprises a costimulatory domain. In a further embodiment, the costimulatory domain comprises the intracellular domain of a costimulatory molecule, selected from CD28, CD27, 4-1BB (CD137), 0X40 (CD134), ICOS (CD278), CD30, CD40, PD-1 (CD279), CD2, CD7, NKG2C (CD94), B7-H3 (CD276) or any combination thereof. In a yet further embodiment, the costimulatory domain comprises the intracellular domain of a costimulatory molecule, selected from CD28, CD27, 4-1BB, OX40, ICOS or any combination thereof, in particular the intracellular domain of 4-1BB.

In one embodiment, the costimulatory domain comprises a 4-1BB signalling domain which has an amino acid sequence with at least 70%, preferably at least 80%, more preferably at least 85%, 90%, 95% 98% or 99% sequence identity with SEQ ID NO: 16. In a further embodiment, the costimulatory domain comprises a 4-1BB signalling domain of SEQ ID NO: 16. This sequence is also found in Uniprot Q07011, residues 214-255.

### Chimeric Antigen Receptor System Arrangements

It will be understood by a person skilled in the art, that various configurations of the domains involved in the CAR described herein are possible, but that the targeting binding domain will necessarily be on the extracellular side of the transmembrane domain and the signalling, costimulatory and HIV Integrase or LEDGF/p75 domains will necessarily be on the intracellular side of the transmembrane domain

In one embodiment, the non-signalling chain comprises domains in the following order: a target binding domain; a transmembrane domain; a costimulatory domain; and an HIV Integrase or LEDGF/p75 domain (arrangement A).

In an alternative embodiment, the non-signalling chain comprises domains in the following order: a target binding domain; a transmembrane domain; the HIV Integrase or LEDGF/p75 domain; and a costimulatory domain (arrangement B).

In another embodiment, the non-signalling chain comprises domains in the following order: a target binding domain; a transmembrane domain and an HIV Integrase or LEDGF/p75 domain (Arrangement C).

In one embodiment, the signalling chain comprises domains in the following order: a transmembrane domain; a costimulatory domain; an HIV Integrase or LEDGF/p75 domain; and a signalling domain (Arrangement I).

In an alternative embodiment, the signalling chain comprises domains in the following order: a transmembrane domain; an HIV Integrase or LEDGF/p75 domain; a costimulatory domain; and a signalling domain (Arrangement II).

In another embodiment, the signalling chain comprises domains in the following order: a transmembrane domain; an HIV Integrase or LEDGF/p75 domain; and a signalling domain (Arrangement III).

Thus, in one embodiment, the CAR comprises a non-signalling chain of Arrangement A, B or C in combination with any signalling chain arranged of Arrangement I, II or III.

It will be understood that in any of these arrangements, either (i) the non-signalling chain comprises an HIV Integrase domain and the signalling chain comprises a LEDGF/p75 domain, or (ii) the non-signalling chain comprises a LEDGF/p75 domain and the signalling chain comprises an HIV Integrase domain.

In another embodiment the CAR comprises a non-signalling chain of Arrangement A with a signalling chain of Arrangement I. In another embodiment the CAR comprises a non-signalling chain of Arrangement A with a signalling chain of Arrangement II. In another embodiment the CAR comprises a non-signalling chain of Arrangement A with a signalling chain of Arrangement III.

In another embodiment the CAR comprises a non-signalling chain of Arrangement B with a signalling chain of Arrangement I. In another embodiment the CAR comprises a non-signalling chain of Arrangement B with a signalling chain of Arrangement II. In another embodiment the CAR comprises a non-signalling chain of Arrangement B with a signalling chain of Arrangement III.

In another embodiment the CAR comprises a non-signalling chain of Arrangement C with a signalling chain of Arrangement I. In another embodiment the CAR comprises a non-signalling chain of Arrangement C with a signalling chain of Arrangement II. In another embodiment the CAR comprises a non-signalling chain of Arrangement C with a signalling chain of Arrangement III.

In one embodiment, the non-signalling chain may comprise a plurality of HIV Integrase or LEDGF/p75 domains. This allows the non-signalling chain to be capable of recruiting more than one signalling chain, and thus amplify the signal in response to target binding. The HIV Integrase or LEDGF/p75 domains may each be variants or fragments with different binding affinities.

In one embodiment, the CAR may comprise two or more target binding domains each recognizing different targets, but comprising the same HIV Integrase or LEDGF/p75 domain. Such a system would be capable of recognizing multiple antigens. In a further embodiment of this arrangement, the HIV Integrase or LEDGF/p75 domain of the receptor components differ in residues which dictate their affinity for the HIV Integrase or LEDGF/p75 domain of the signalling chain. In this way, the CAR can be tuned such that signalling in response to one antigen is greater or lesser than the response to another.

In one embodiment, the CAR described herein may comprise a plurality of signalling chains, each comprising a signalling domain and a HIV Integrase or LEDGF/p75 domain, wherein the signalling chains comprise different signalling domains (e.g. CD3zeta, CD28, 4-1BB and/or OX-40). This allows the activation of multiple different signalling domains simultaneously.

Methods suitable for altering the amino acid residues of the HIV Integrase or LEDGF/p75 domains such that the binding affinity between the two domains is altered are known in the art. For example, such methods include substitution, addition and removal of amino acids using both targeted and random mutagenesis.

Methods for determining the binding affinity between the HIV Integrase and LEDGF/p75 domains are also well known in the art. These include bioinformatics prediction of protein-protein interactions, affinity electrophoresis, surface plasma resonance, bio-layer interferometry, dual polarisation interferometry, static light scattering and dynamic light scattering.

### Polynucleotides and Expression Vectors

According to a further aspect of the invention, there is provided a polynucleotide encoding the signalling chain, a polynucleotide encoding the non-signalling chain or a polynucloeotide chain encoding the signalling and non-signalling chains of the CAR described herein.

The polynucleotide sequences described herein may be codon optimised. The degeneracy found in the genetic code allows each amino acid to be encoded by between one and six synonymous codons allowing many alternative nucleic acid sequences to encode the same protein (Gustafsson et al. (2004) Trends Biotechnol. 22(7): 346-53). Codon optimisation is a technique used to modify genetic sequences with the intent of increasing the rate of expression of a gene in a heterologous expression system; typically the nucleotide sequence encoding a protein of interest is codon optimized such that the codon usage more closely resembles the codon bias of the host cell, while still coding for the same amino acid sequence.

Nucleic acids described herein may comprise DNA or RNA. They may be single-stranded or double-stranded. They may also be polynucleotides which include within them synthetic or modified nucleotides. A number of different types of modification are well known in the art, such as methylphosphonate and phosphorothioate backbones, or addition of acridine or polylysine chains. Such modifications can be used in order to enhance in vivo activity or life span of the polynucleotides of the present invention.

The signalling chain and the non-signalling chain of the CAR may be expressed by separate nucleic acids or co-expressed from the same nucleic acid.

Methods of simultaneous expression of more than one gene in cells or organisms using a single plasmid are well known in the art. For example, methods include: multiple promoters fused to the genes' open reading frames (ORFs); insertion of splicing signals between genes; fusion of genes whose expressions are driven by a single promoter; insertion of proteolytic cleavage sites between genes; insertion of internal ribosomal entry sites (IRESs) between genes; insertion of self-cleaving peptide sequences between genes.

If the components are co-expressed, the nucleic acid may produce a polypeptide which comprises the signalling chain and the non-signalling chain joined by a cleavage site. The cleavage site may be self-cleaving such that when the polypeptide is produced, it is immediately cleaved into the signalling chain and the non-signalling chain component without the need for any external cleavage activity. Therefore, according to a further aspect of the invention, there is provided a polynucleotide encoding a CAR described herein, wherein the signalling chain and the non-signalling chain are co-expressed by means of a self-cleaving peptide which is cleaved between the signalling chain and the non-signalling chain after translation.

Examples of self-cleaving peptides are known in the art, for example see Kim et al. (2011) PLoS ONE 6(4): e18556.

In one embodiment, the self-cleaving peptide is a 2a self-cleaving peptide. In a further embodiment, the 2a self-cleaving peptide is derived from porcine teschovirus-1, Thosea asigna virus, equine rhinitis A virus (ERAV) or foot-and-mouth disease virus (FMDV), in particular porcine teschovirus-1. Therefore, the self-cleaving peptide may be selected from: P2A (porcine teschovirus-1 2A), T2A (Thosea asigna virus 2A), E2A (equine rhinitis A virus 2A) and F2A (foot-and-mouth disease virus 2A). In a yet further embodiment, the 2a self-cleaving peptide comprises SEQ ID NO: 17.

In one embodiment, there is a linker between the signalling chain and the self-cleaving peptide and/or between the non-signalling chain and the self-cleaving peptide. In a further embodiment, the linker comprises SEQ ID NO: 8 or 9.

In one embodiment, the components are co-expressed using a polynucleotide comprising a co-expressing sequence. In a further embodiment, the co-expressing sequence is an internal ribosome entry site (IRES) or an internal promoter.

The polynucleotide may be present in an expression cassette or expression vector (e.g. a plasmid for introduction into a bacterial host cell, or a viral vector such as a lentivirus for transfection of a mammalian host cell). Therefore, according to a further aspect of the invention, there is provided an expression vector comprising the polynucleotide described herein.

The term “vector” refers to a vehicle which is able to artificially carry foreign genetic material into another cell, where it can be replicated and/or expressed. In one embodiment, the vector is a plasmid, a viral vector, a transposon based vector or a synthetic mRNA.

In one embodiment, the expression vector is a retroviral vector. In a further embodiment, the retroviral vector is derived from, or selected from, a lentivirus, alpha-retrovirus, gamma-retrovirus or foamy-retrovirus, such as a lentivirus or gamma-retrovirus, in particular a lentivirus. In a further embodiment, the retroviral vector particle is a lentivirus selected from the group consisting of HIV-1, HIV-2, SN, FIV, EIAV and Visna. Lentiviruses are able to infect non-dividing (i.e. quiescent) cells which makes them attractive vectors for gene therapy. In a yet further embodiment, the retroviral vector particle is HIV-1 or is derived from HIV-1. The genomic structure of some retroviruses may be found in the art. For example, details on HIV-1 may be found from the NCBI Genbank (Genome Accession No. AF033819). HIV-1 is one of the best understood retroviruses and is therefore often used as a viral vector.

### Immunomodulatory Cells

According to a further aspect of the invention, there is provided an immunomodulatory cell comprising the CAR described herein. According to another aspect of the invention, there is provided an immunomodulatory cell comprising a polynucleotide or expression vector described herein.

In one embodiment, the immunomodulatory cell comprises a signalling chain and a non-signalling chain of the CAR described herein. In a further embodiment, the immunomodulatory cell comprises a number of different signalling chains and a number of different non-signalling chains. For example, the immunomodulatory cell may comprise one, two, three, four, five or more different signalling chains and one, two, three, four, five or more different non-signalling chains of the CAR described herein.

The term “immunomodulatory cell” refers to a cell of hematopoietic origin functionally involved in the modulation (e.g. the initiation and/or execution) of the innate and/or adaptive immune response. Said immunomodulatory cell according to the present invention can be derived from a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells. Said immunomodulatory cell can also be a dendritic cell, a killer dendritic cell, a mast cell, a NK-cell, a B-cell or a T-cell. The T-cell may be selected from the group consisting of inflammatory T-lymphocytes, cytotoxic T-lymphocytes, regulatory T-lymphocytes or helper T-lymphocytes, or a combination thereof. Therefore, in one embodiment, the immunomodulatory cell is derived from an inflammatory T-lymphocyte, cytotoxic T-lymphocyte, regulatory T-lymphocyte or helper T-lymphocyte. In another embodiment, said cell can be derived from the group consisting of CD4⁺ T-lymphocytes and CD8⁺ T-lymphocytes.

In one embodiment, the immunomodulatory cell may be a human immunomodulatory cell.

In one embodiment, the immunomodulatory cell is allogeneic or autologous. It will be understood that “autologous” refers to cells obtained from the patient themselves, whereas “allogeneic” refers to cells obtained from a donor. Autologous cells have the advantage that they are compatible with the patient and therefore avoid any immunological compatibility problems leading to graft-versus-host disease (GvHD). In order to prevent the allogeneic cells from being rejected by the patient, they would either need to be derived from a compatible donor or modified to ensure no antigens are present on the cell surface which would initiate an unwanted immune response.

Prior to expansion and genetic modification of the cells of the invention, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumours. In certain embodiments of the present invention, any number of T cell lines available and known to those skilled in the art, may be used. In another embodiment, said cell can be derived from a healthy donor or a diseased donor, such as a patient diagnosed with cancer or an infection. In another embodiment, said cell is part of a mixed population of cells which present different phenotypic characteristics.

The immunomodulatory cells may be activated and/or expanded prior to being transduced with polynucleotides or expression vectors encoding the CARs described herein. For example, the cells may be treated with an anti-CD3 monoclonal antibody to cause activation.

It will be understood that the immunomodulatory cells may express the CARs described herein transiently or stably/permanently (depending on the transfection method used and whether the polynucleotide encoding the chimeric antigen receptor system has integrated into the immunomodulatory cell genome or not).

After introduction of the CAR, the immunomodulatory cells may be purified, for example by selecting cells expressing the target binding domain of the non-signalling chain.

### Uses

According to a further aspect of the invention, there is provided the immunomodulatory cell described herein for use in therapy. In one embodiment, therapy comprises administration of the immunomodulatory cell to a human subject in need of such therapy.

In one embodiment, the therapy is adoptive cellular therapy. “Adoptive cellular therapy” (or “adoptive immunotherapy”) refers to the adoptive transfer of human T lymphocytes that are engineered by gene transfer to express CARs (such as the CARs of the present invention) specific for surface molecules expressed on target cells. This can be used to treat a range of diseases depending upon the target chosen, e.g. tumour specific antigens to treat cancer. Adoptive cellular therapy involves removing a portion of the patient's white blood cells using a process called leukapheresis. The T cells may then be expanded and mixed with expression vectors described herein in order to permanently transfer the CAR to the T cells. The T cells are expanded again and at the end of the expansion, the T cells are washed, concentrated, and then frozen to allow time for testing, shipping and storage until the patient is ready to receive the infusion of engineered T cells.

The invention described herein provides for the first time the use of the HIV Integrase-LEDGF/p75 interaction in a CAR. Therefore, according to a further aspect of the invention there is provided the use of a LEDGF/p75 domain and HIV Integrase domain, or functional fragments or variants thereof, as a safety switch in a chimeric antigen receptor (CAR) T cell therapy (e.g. as part of an inducible CAR).

### PHARMACEUTICAL COMPOSITIONS

According to a further aspect of the invention, there is provided a pharmaceutical composition comprising a plurality of immunomodulatory cells as defined herein.

Examples of additional pharmaceutical composition ingredients include, without limitation, any adjuvants, carriers, excipients, glidants, sweetening agents, diluents, preservatives, dyes/colourants, flavour enhancers, surfactants, wetting agents, dispersing agents, suspending agents, stabilizers, isotonic agents, solvents, surfactants, emulsifiers, buffers (such as phosphate buffered saline (PBS)), carbohydrates (such as glucose, mannose, sucrose or dextrans), amino acids, antioxidants or chelating agents (such as EDTA or glutathione).

In one embodiment, the pharmaceutical composition additionally comprises a pharmaceutically acceptable excipient, carrier, or diluent. The carrier, excipient or diluent must be “acceptable” in the sense of being compatible with the other ingredients of the composition and not deleterious to the recipient thereof. According to the present invention any excipient, vehicle, diluents or additive used would have to be compatible with the CAR described herein. Standard texts known in the art, such as “Remington's Pharmaceutical Science”, 17th Edition, 1985, incorporated herein by reference, may be consulted to prepare suitable preparations.

Pharmaceutical compositions may be administered by injection or continuous infusion (examples include, but are not limited to, intravenous, intratumoural, intraperitoneal, intradermal, subcutaneous, intramuscular and intraportal). In one embodiment, the composition is suitable for intravenous administration. When administering a therapeutic composition of the present invention (e.g., a pharmaceutical composition containing a genetically modified immunomodulatory cell as described herein), it will generally be formulated in a unit dosage injectable form (solution, suspension, emulsion). Pharmaceutical compositions may be suitable for topical administration (which includes, but is not limited to, epicutaneous, inhaled, intranasal or ocular administration) or enteral administration (which includes, but is not limited to, oral or rectal administration).

Methods for the preparation of such pharmaceutical compositions are well known to those skilled in the art. Other excipients may be added to the composition as appropriate for the mode of administration and the particular protein used.

Effective doses and treatment regimens for administering the composition of the present invention may be dependent on factors such as the age, weight and health status of the patient and disease to be treated. Such factors are within the purview of the attending physician.

According to a further aspect of the invention, there is provided a pharmaceutical composition as defined herein, for use in the treatment or prevention of a disease.

In one embodiment, the disease is selected from: a cancer, a pathogenic immune response and an infection.

According to a further aspect of the invention, there is provided the use of a pharmaceutical composition as described herein, in the manufacture of a medicament for the treatment and/or prevention of a disease.

### Kits

According to a further aspect of the invention, there is provided a kit which comprises a polynucleotide or expression vector as described herein.

According to a further aspect of the invention, there is provided a kit which comprises an immunomodulatory cell or pharmaceutical composition as described herein.

According to a further aspect of the invention, there is provided a kit which comprises the CAR as described herein.

### Methods

According to a further aspect of the invention, there is provided a method of engineering an immunomodulatory cell to express the CAR described herein, comprising:
(a) providing an immunomodulatory cell;
(b) transducing or transfecting the polynucleotide or the expression vector as defined herein, into said immunomodulatory cell; and
(c) expressing said polynucleotide or said expression vector in the immunomodulatory cell.

In one embodiment, the immunomodulatory cell is obtained from a sample isolated from a patient (i.e. autologous). In an alternative embodiment, the immunomodulatory cell is obtained from a donor (i.e. allogeneic).

As a non-limiting example, the CAR can be introduced as transgenes encoded by an expression vector as described herein. The expression vector can also contain a selection marker which provides for identification and/or selection of cells which received said vector.

Polypeptides may be synthesized in situ in the cell as a result of the introduction of polynucleotides encoding said CAR into the cell. Alternatively, said polypeptides could be produced outside the cell and then introduced thereto. Methods for introducing a polynucleotide construct into cells are known in the art and including, as non-limiting examples, stable transformation methods wherein the polynucleotide construct is integrated into the genome of the cell or transient transformation methods wherein the polynucleotide construct is not integrated into the genome of the cell and virus mediated methods. Said polynucleotides may be introduced into a cell by, for example, recombinant viral vectors (e.g. retroviruses, adenoviruses), liposomes and the like. For example, transient transformation methods include for example microinjection, electroporation or particle bombardment. The polynucleotides may be included in vectors, more particularly plasmids or viruses, in view of being expressed in cells.

The terms “transfection”, “transformation” and “transduction” as used herein, may be used to describe the insertion of the expression vector into the target cell. Insertion of a vector is usually called transformation for bacterial cells and transfection for eukaryotic cells, although insertion of a viral vector may also be called transduction. The skilled person will also be aware of the different non-viral transfection methods commonly used, which include, but are not limited to, the use of physical methods (e.g. electroporation, cell squeezing, sonoporation, optical transfection, protoplast fusion, impalefection, magnetofection, gene gun or particle bombardment), chemical reagents (e.g. calcium phosphate, highly branched organic compounds or cationic polymers) or cationic lipids (e.g. lipofection). Many transfection methods require the contact of solutions of plasmid DNA to the cells, which are then grown and selected for a marker gene expression.

Once the CAR has been introduced into the immunomodulatory cell, said cell may be referred to as a “transformed immunomodulatory cell”. Therefore, according to a further aspect of the invention, there is provided an immunomodulatory cell obtained by the method described herein. Also within the scope of the present invention is a cell line obtained from a transformed immunomodulatory cell according to the method described herein.

According to a further aspect of the invention, there is provided a method of inhibiting a CAR in a subject, which comprises administering to the subject an agent that inhibits the LEDGF/p75-HIV Integrase domain interaction.

The level of CAR signalling by the system described herein, may be adjusted by altering the amount of disrupting agent present, or the amount of time the disrupting agent is present. Therefore, in one embodiment, the level of CAR cell activation may be increased by decreasing the dose of disrupting agent administered to the subject or decreasing the frequency of its administration. In an alternative embodiment, the level of CAR cell activation may be reduced by increasing the disrupting dose of the agent, or the frequency of administration to the subject.

Higher levels of CAR signalling are likely to be associated with reduced disease progression but increased toxic activities, whilst lower levels of CAR signalling are likely to be associated with increased disease progression but reduced toxic activities.

According to a further aspect of the invention, there is provided a method of treating and/or preventing a disease, which comprises administering to a subject the immunomodulatory cell or the pharmaceutical composition as defined herein.

In one embodiment, the disease is cancer. In a further embodiment, the cancer is selected from: blood, bone marrow, lymph, lymphatic system, bladder, breast, colon, cervix, esophagus, kidney, large intestine, lung, oral cavity, ovary, pancreas, prostate, rectum, skin or stomach. In a yet further embodiment, the cancer is a blood cancer, for example selected from the group consisting of: B cell leukaemia, multiple myeloma (MM), acute lymphoblastic leukaemia (ALL), chronic lymphocytic leukaemia (CLL) and non-Hodgkin's lymphoma.

When the method described herein is used to treat cancer, in one embodiment, the method reduces the number of tumour cells, reduces the tumour size and/or eradicates the tumour in the subject.

In one embodiment, the disease is a pathogenic immune response, such as an autoimmune disease, allergy or graft-versus-host rejection. Autoimmune diseases arise from an abnormal immune response of the body against substances and tissues normally present in the body. This can result in the damage or destruction of tissues, or altered organ growth or function. Examples of autoimmune diseases include, but are not limited to: diabetes mellitus Type 1, arthritis (including juvenile, psoriatic, reactive, and rheumatoid arthritis), psoriasis, multiple sclerosis, vasculitis, alopecia areata, pernicious anaemia, glomerulonephritis, autoimmune hepatitis, autoimmune pancreatitis, ulcerative colitis, systemic lupus erythematosus, Graves' disease, Guillain-Barre syndrome, Sjogren's syndrome, Celiac disease, Crohn's disease and Wegener's syndrome.

In one embodiment, the disease is an infection. An infection can be caused by a pathogen, such as a bacteria, virus, parasite, protozoa or fungi. In a further embodiment, the infection is a viral or bacterial infection.

In one embodiment the subject is a human.

The method of treatment and/or prevention, may comprise the following steps:
(a) providing an immunomodulatory cell(s);
(b) transducing or transfecting the polynucleotide or the expression vector as defined herein, into said immunomodulatory cell(s);
(c) expressing said polynucleotide or said expression vector in the immunomodulatory cell(s); and
(d) administering the immunomodulatory cell(s) to a patient.

The immunomodulatory cells or pharmaceutical compositions described herein may be administered to a patient who already has the disease in order to lessen, reduce or improve at least one symptom associated with the disease and/or to slow down, reduce or block the progression of the disease (i.e. therapeutically). The immunomodulatory cells or pharmaceutical compositions described herein may be administered to a patient who has not yet contracted the disease and/or who is not showing any symptoms of the disease to prevent the cause of the disease (i.e. prophylactically). The patient may have a predisposition for, or be thought to be at risk of developing the disease.

In one embodiment, the method additionally comprises administering an agent which disrupts the interaction between the LEDGF/p75—and HIV Integrase domains of the CAR described herein. The agent may be administered to the patient before or simultaneously with the immunomodulatory cells/pharmaceutical composition (i.e. prior to or during step (d) in the method of treatment steps outlined above) in order to administer the CAR in its “inactive” (i.e. OFF) state. The amount of agent can then be decreased in order to activate the CAR. Administering the CAR in its inactive state allows for an even distribution of the immunomodulatory cells to be achieved, therefore preventing local accumulation of activated cells.

Alternatively, the agent may be administered to the patient after administration of the immunomodulatory cells/pharmaceutical composition (i.e. after step (d) in the method of treatment steps outlined above) so that the CAR is administered in its “active” (i.e. ON) state.

The agent may be administered in the form of a pharmaceutical composition. In this embodiment, the composition may additionally comprise pharmaceutically acceptable carriers, diluents or excipients as outlined herein.

As described herein, the present invention provides a reversible OFF-switch to be used with CAR-T cell therapies. The method may involve monitoring toxic activity in the patient. Thus, if the level of toxic activity becomes too high, the method can involve administering an agent which inhibits the LEDGF/p75-HIV Integrase interaction to reduce adverse toxic side effects. Toxic activities include, for example, immunological toxicity, biliary toxicity and respiratory distress syndrome.

Similarly, the method may involve monitoring the progression of disease and then administering an agent which inhibits the LEDGF/p75-HIV Integrase interaction when an acceptable level of disease progression is reached (e.g. amelioration). The specific level of disease progression determined to be “acceptable” will vary according to the specific circumstances and should be assessed on such a basis.

Monitoring the progression of the disease means to assess the symptoms associated with the disease over time to determine if they are reducing/improving or increasing/worsening.

According to a further aspect of the invention, there is provided an agent suitable for inhibiting the CAR as defined herein.

It will be understood that the embodiments described herein may be applied to all aspects of the invention. Furthermore, all publications, including but not limited to patents and patent applications, cited in this specification are herein incorporated by reference as though fully set forth.

### EXAMPLES

### Example 1

### Construction of Chimeric Antigen Receptors that Incorporate Domains of HIV Integrase and Human LEDGF/p75 Acting as an OFF-Switch Control Element

CARs prepared with LEDGF/p75 and HIV Integrase elements are capable of activating T-cell NFAT signalling cascade in an antigen-specific manner provided that both signalling and antigen-binding chains are tethered to the cell membrane. The NFAT activation signal of Jurkat cells transfected with the OFF-switch CAR are modulated by the presence of LEDGIN compound BI224436.

### Materials and Methods

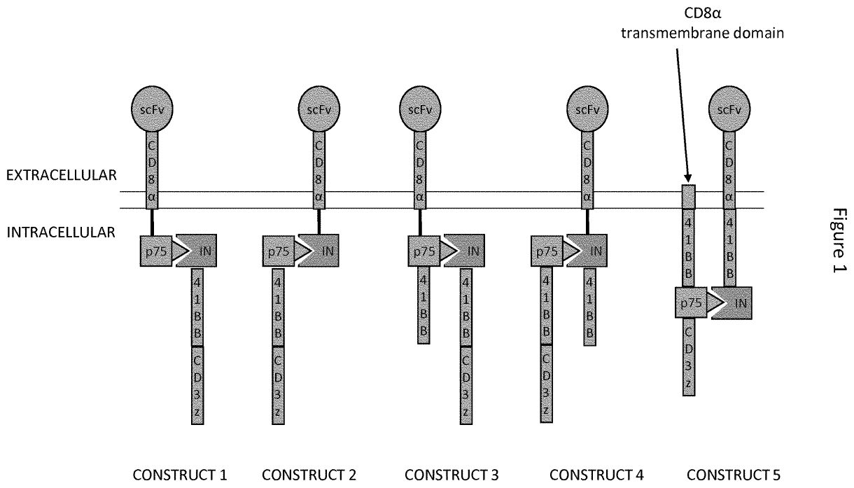

Generation of constructs: Constructs below were cloned into pG3 vector. Schematic representation of constructs is shown in FIG. 1 and full sequence details of the constructs are given below.

Transfection of Jurkat Cells with Constructs and NFAT-Luciferase Assay

NFAT-luc2 Jurkat cells (Promega) were cultured in Jurkat media: RPMI medium 1640 (1×) without L-glutamine with phenol red (Gibco), 10% (v/v) Fetal Bovine Serum (FBS) Heat-Inactivated (Gibco), 1% (v/v) Minimum essential medium non-essential amino acids (MEM NEAA) (ThermoFisher), 1% (v/v) Sodium Pyruvate (Sigma), 1% (v/v) L-Glutamine (Gibco). ARH-77-10B5 cells were cultured in Jurkat media plus 1 mg/mL G418 (Thermo, 10131035). Cells were incubated at 37° C. with 5% CO₂ for 48 hours. 20 μg of plasmid DNA from each construct was mixed with 8×10⁶ NFAT-luc2 Jurkat cells and cells were transfected using the 4D-Nucleofector (Lonza) with cell Line SE Nucleofector kit (Lonza) by following manufacturer instructions with program CL-120. BI224436 compound (Chemexpress HY-18595) at a stock concentration of 100 mM in 100% DMSO (Sigma, D2650) was diluted in Jurkat media to achieve a stock concentration of 100 μM. Using the 100 μM BI224436 stock in Jurkat media, NFAT-luc2 Jurkat cells were incubated at a final compound concentration of 3 μM for 48 hours at 37° C. with 5% CO₂. NFAT-luc2 Jurkat cells were then co-cultured (1×10⁵ cells per well, 1:1 effector:target ratio) with ARH-77-10B5 cells (BCMA positive cells) for 6.5 hours. NFAT-luciferase luminescence was measured by using the Bio-Glo Luciferase Assay System (Promega G7940) (following manufacturer's instructions) and an EnSpire Multimode Plate Reader (Perkin Elmer).

### Results

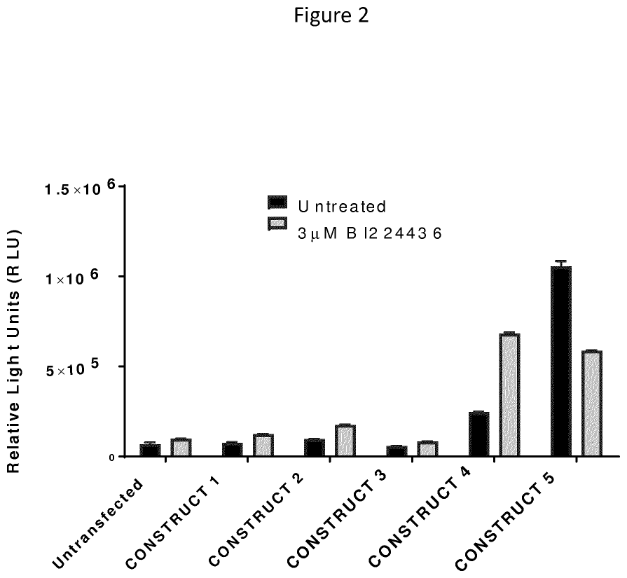

Constructs 1 to 4 encode for OFF-switch CARs in which the target-binding domain (anti-BCMA scFv) is fused to either a human LEDGF/p75 domain or an HIV integrase domain (FIG. 1). In these constructs, the signalling chain is not tethered to the plasma membrane. Constructs 1 to 4 show no or low NFAT activation when stimulated with antigen-presenting cells and addition of BI224436 (a small molecule binder of HIV integrase that inhibits its binding to LEDGF/p75) does not reduce the level of activation (FIG. 2). Construct 5 encodes for an OFF-switch CAR in which the signalling chain is tethered to the plasma membrane via fusion to a CD8α transmembrane domain. Construct 5 activates NFAT in Jurkat cells when presented with antigen-expressing cells (ARH-77-10B5) and NFAT activation is reduced in the presence of BI224436.

### Example 2

### Activation of Chimeric Antigen Receptor Containing Off-Switch Elements is Improved by the Incorporation of an HIV Integrase Solubilising Mutation

The capacity of the LEDGF/p75 and HIV Integrase OFF-switch CAR to activate the NFAT promoter in an antigen-specific fashion can be improved by the introduction of one HIV integrase mutation described in Jenkins et. al. (1995) PNAS Vol. 92, pp 6057-6061. This mutation is the substitution of Phenyalanine 185 to Lysine (F185K) in the integrase domain. The mutation corresponds to Phenylalanine 1332 on the HIV Gag-Pol polyprotein with Uniprot entry P12497.

Generation of constructs: Construct 5 was generated in Example 1. Construct below was cloned into pG3 vector. Full sequence details of the construct is given below.

Transfection of Jurkat cells with Constructs and NFAT-Luciferase Assay

Method is described on Example 1.

### Results

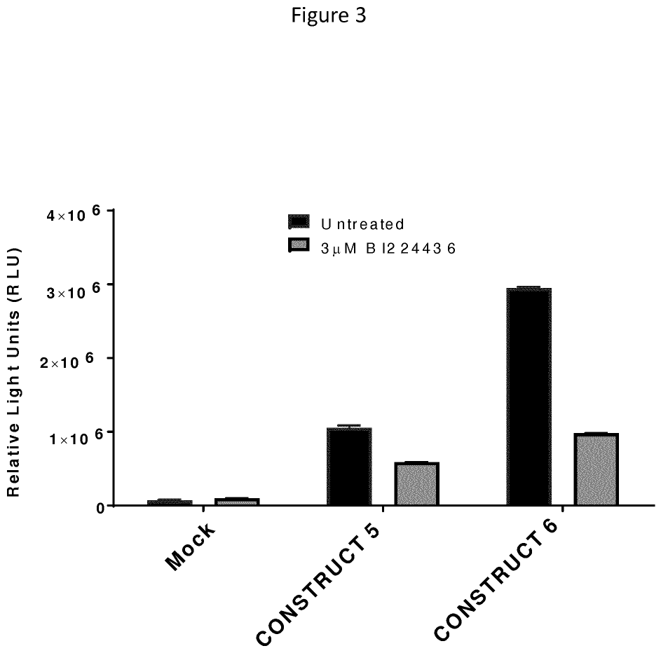

The mutation of the wild type HIV integrase Phenylalanine 1332 (Uniprot entry P12497) to Lysine in Construct 6 results in a three-fold increase in the activation of the NFAT promoter in Jurkat cells with respect to Construct 5 (FIG. 3). The effect of compound BI224436 on the downregulation of NFAT is preserved in both Constructs 5 and 6.

### Example 3

### Activation of Chimeric Antigen Receptor Containing OFF-Switch Elements is Improved by Swapping the LEDGF/p75 Domain to the Antigen-Binding Chain.

The capacity of the LEDGF/p75 and HIV Integrase OFF-switch CAR to activate the NFAT promoter can be further improved by having the LEDGF/p75 domain fused to the antigen-binding chain and the HIV integrase domain fused to the signalling chain.

Generation of constructs: Construct 6 was generated in Example 2. Construct below was cloned into pG3 vector. Full sequence details of the construct is given below.

Transfection of Jurkat cells with constructs and NFAT-luciferase assay:Method is described on Example 1.

### Results

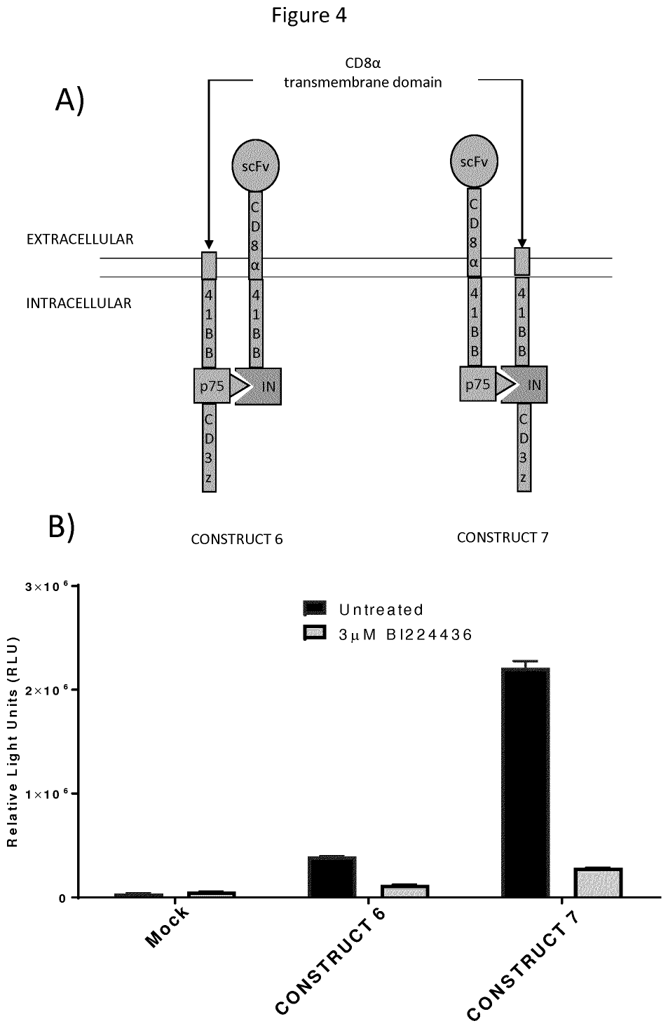

NFAT activation is more than 4-fold higher in Construct 7, where the LEDGF/p75 domain is fused to the antigen-binding chain and HIV integrase domain is fused to the signalling chain, compared with Construct 6, where the HIV integrase domain is fused to the antigen-binding chain and LEDGF/p75 is fused to the signalling chain (FIG. 4). The effect of compound BI224436 on the downregulation of NFAT is preserved in both Constructs 6 and 7.

### Example 4

### Expression Levels of CAR with OFF-Switch Elements is Improved by Changes to the Human LEDGF/p75 Domain

Expression levels of the OFF-switch CAR can be improved by mutating the LEDGF/p75 domain. Five LEDGF/p75 engineered variants were generated: three variants include the C-terminal addition of unstructured (based on the PDB entry 1Z9E) wild type LEDGF/p75 sequence to Construct 7 and the other two variants include novel point mutations that enhanced expression of the OFF-switch CAR.

Generation of constructs: Construct 7 was generated in Example 3. Constructs below were cloned into pG3 vector. Full sequence details of the constructs are given below.

Lentiviral vector production, transduction of Jurkat cells and flow cytometry: For lentiviral vector production, 3.0×10⁷ Lenti-X 293T (Takara Bio) cells were seeded in 20 mL DMEM (Gibco) and were incubated overnight at 37° C. with 5% CO₂. Lenti-X 293T cells were transfected by mixing 21 μg of transfer vector containing the construct, 3.75 μg ViraSafe pRSV-Rev, 5.25 μg ViraSafe pCMV-VSVG, 7.5 μg ViraSafe pCgp V-(gag-pol), 75 pg jetPRIME (Polyplus) and 1500 μg jetPRIME Buffer (Polyplus). After 2 days, supernatants were clarified and virus was concentrated and purified by ultracentrifugation on a 20% sucrose cushion using Ultrapure sucrose (ThermoFisher) in 50 ml Oak Ridge PPCO ultracentrifugation tubes (ThermoFisher). Lentiviral vectors were produced for CONSTRUCT 7, 8 ,9 10, 11 and 12 the method described above.

Jurkat cells were grown as described in Example 1. Prior to transduction, cells were split to a density of 2×10⁵ cells/ml. Jurkats were transduced with the lentiviral vectors encoding for CONSTRUCT 7, 8, 9, 10, 11, and 12. Transduction reactions were prepared to achieve an MOI of 5. Transduced Jurkat cells were cultured at a density of 2×10⁵ cells/ml for 20 days. At 20 days after transduction, cells were stained with AlexaFluor 647 conjugated BCMA-Fc (generated in-house) to label the anti-BCMA scFv domain on the CARs . Measurements were made using a Cytoflex S (Beckman Coulter) and data analysed using FlowJo (FlowJo).

### Results

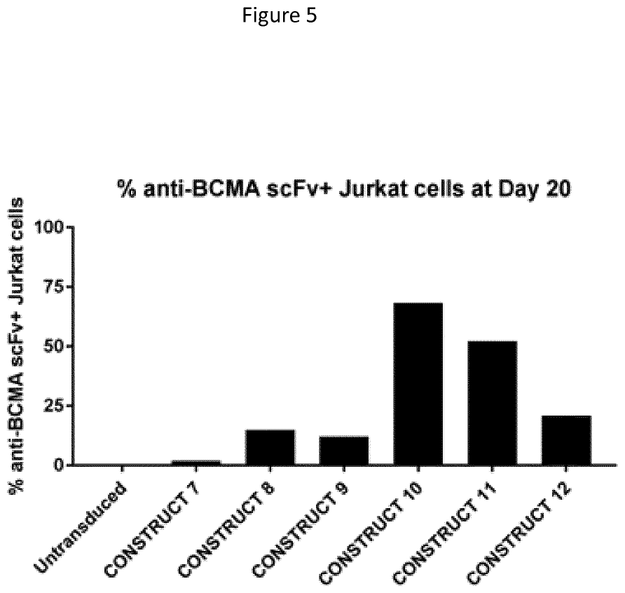

Extension of the C-terminal portion of LEDGF/p75 (Constructs 8, 9, and 10) and mutation of LEDGF/p75 residues 428 and 429 (Constructs 11 and 12), resulted in an increase of the percentage of cells expressing the HIV LEDGF/p75 OFF-CAR (FIG. 5).

### Example 5

### Cytokine Release by Primary T-Cells Transduced with the HIV Integrase LEDGF/p75 OFF-CAR.

Primary T-cells transduced with the HIV Integrase LEDGF/p75 OFF-CAR produce a functional response in the form of cytokine release when stimulated with antigen-presenting cells. Addition of BI224436 negatively modulates the cytokine release.

### Materials and Methods

Primary T-cell transduction and cytokine release: Peripheral blood mononuclear cells (PBMCs) from the fresh blood of two healthy human donors were isolated by density gradient centrifugation in Accuspin tubes (Sigma) containing 15 mL of Histopaque-1077 (Sigma) and following manufacturer's instructions. Cells were resuspended at 1×10⁶ cells/mL in TEXMacs media (Miltenyi Biotec) containing 100 units/mL of IL-2 (Sigma) and TransAct beads (Miltenyi Biotec) and incubated for 48 hours at 37° C. with 5% CO₂.

T-cells from the two donors were transduced with the lentiviral vectors encoding for CONSTRUCT 10 and CONSTRUCT 11. Transduction reactions were prepared to achieve an MOI of 5. T-cells were cultured in TEXMacs media (Miltentyi Biotec) with 100 units/mL of IL-2, fresh media was added every 3 days. ARH-77-10B5 cells were cultured as described in Example 1. BI224436 compound at a stock concentration of 100 mM in 100% DMSO was diluted in Jurkat media to achieve a stock concentration of 100 μM. Each T-cell population was incubated at a final concentration of 10 μM or 0 μM (media alone) BI224436 in Jurkat media for 1 hour at 37° C. with 5% CO₂. T-cells were then co-cultured (5×10⁴ cells per well, 1:1 effector:target ratio) with either ARH-77-10B5 cells (BCMA positive cells) or media (no antigen) for 24 hour in Jurkat media at 37° C. with 5% CO₂. Cells were pelleted (1200 rpm, 5 min) and supernatants were collected. Supernatants were analysed for cytokine levels using MSD V-plex Proinflammatory Panel 1 Human Kit (MSD, K15049D-2) and MSD Sector Imager (MSD).

### Results

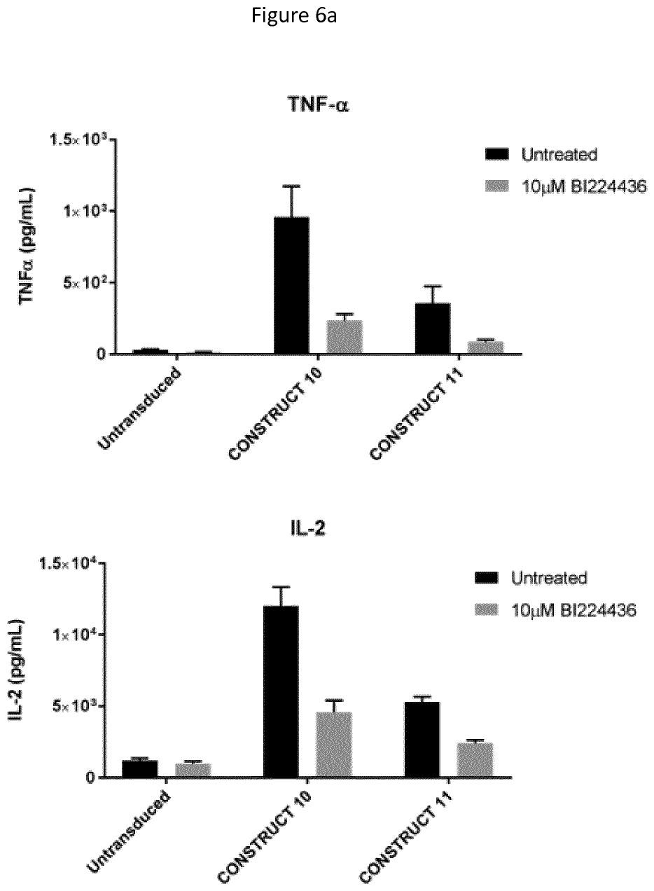

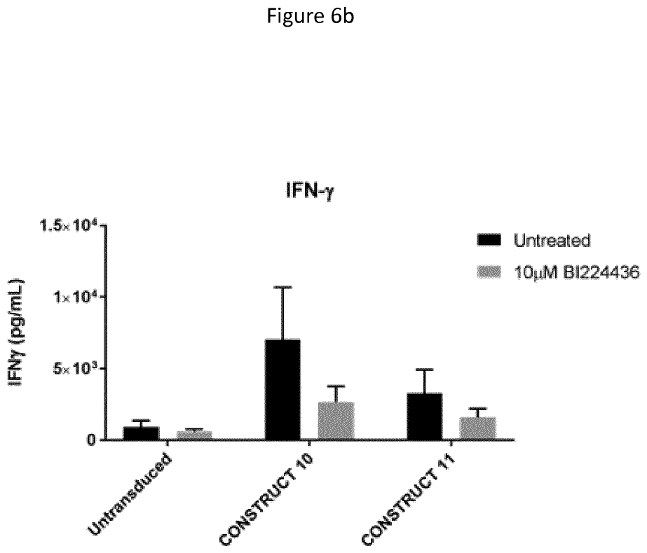

Primary T-cells transduced with OFF-switch CARs (Construct 10 and 11) release cytokines TNF-alpha, IL-2 and IFN-gamma upon stimulation with BCMA positive cells (ARH-77-10B5) (FIG. 6). In all cases, cytokine release was attenuated by the addition of the BI224435 compound.

### Example 6

### Tuning the Level of Cytokine Release by Primary T-cells Transduced with the HIV Integrase LEDGF/p75 OFF-CAR

The cytokine release of primary T-cells transduced with the HIV Integrase LEDGF/p75 OFF-switch CAR can be tuned by the concentration of BI224436. An OFF-switch CAR with a Myc-tag on the signalling chain was used.

### Materials and Methods

Generation of constructs: Construct 13 was cloned into pG3 vector. Full sequence details of the construct is given below.

Lentiviral vector production and transduction of primary T-cells: Method for lentiviral production is described in Example 4 and method for primary T-cell transduction is described in Example 5.

Cytokine release: ARH-77-10B5 cells were cultured as described in Example 1. BI224436 compound at a stock concentration of 100 mM in 100% DMSO was diluted in Jurkat media to achieve a stock concentration of 100 μM. Each T-cell population was incubated at a final concentration of 10 μM, 3.3 μM, 1.1 μM, 0.37 μM, 0.12 μM, 0.04 μM, 0.013 μM, 0.004 μM, 0.001 μM or 0 μM (media alone) BI224436 in Jurkat media for 1 hour at 37° C. with 5% CO₂. T-cells were then co-cultured (5×10⁴ cells per well, 1:1 effector:target ratio) with ARH-77-10B5 cells (BCMA positive cells) for 24 hours in Jurkat media at 37° C. with 5% CO₂. Cells were pelleted (1200 rpm, 5 min) and supernatants were collected. Supernatants were analysed for cytokine levels using MSD V-plex Proinflammatory Panel 1 Human Kit (MSD, K15049D-2) and MSD Sector Imager (MSD).

### Results

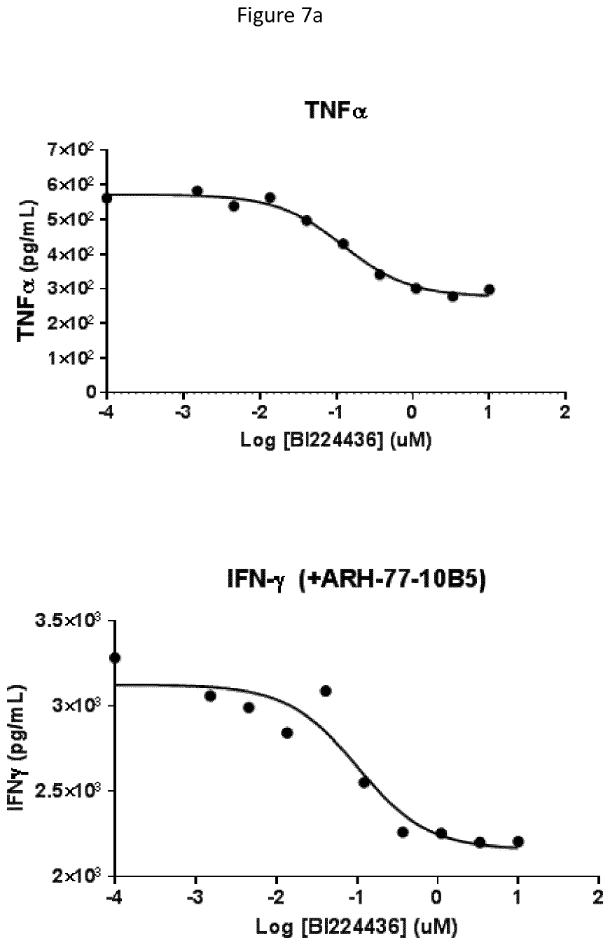

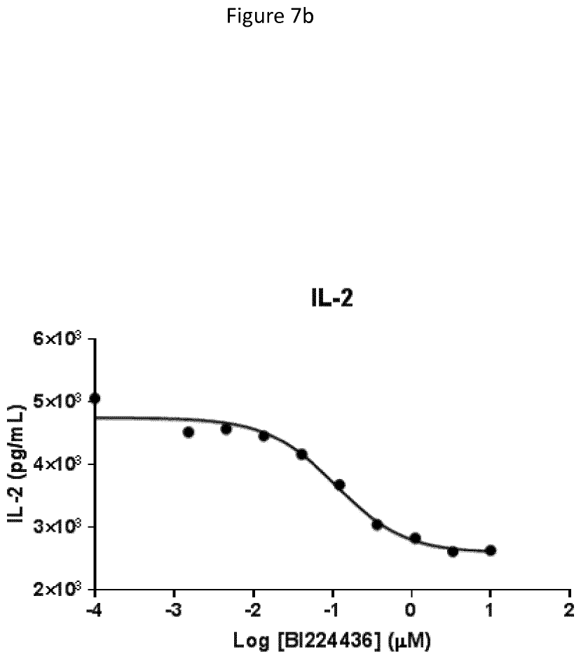

The concentration of BI224436 tunes the level of TNF-alpha, IL-2 and IFN-gamma release from primary T-cells transduced with the OFF-switch CAR (Construct 13), when stimulated with BCMA positive cells (ARH-77-10B5) presenting cells (FIG. 7). The IC50 concentration of BI224436 is 100 nM. This concentration is 10-fold lower than the BI224436 concentrations achieved in the plasma of human subjects 24 hours after 200 mg oral doses of BI224436 (Clinical trial ID NCT01276990, analysis presented as a conference report http://www.natap.org/2011/ICAAC/ICAAC_35.htm).

### Example 7

### Cytotoxicity by Primary T-Cells Transduced with the HIV Integrase LEDGF/p75 OFF-Switch CAR.

The HIV Integrase LEDGF/p75 OFF-switch CAR activates a cytotoxic response and cytotoxicity is attenuated by the presence of compound BI224436.

### Materials and Methods

Transduction of primary T-cells: Method described in Example 5

xCelligence cytotoxicity assay ARH-77-10B5 cells were as described in Example 1. BI224436 compound at a stock concentration of 100 mM in 100% DMSO was diluted in Jurkat media to achieve a stock concentration of 100 μM. xCelligence E-plates (ACEA, 06472460001) were coated with anti-CD40 tethering agent by following manufacturer's instructions for B-cell Killing (anti-CD40) Assay kit (ACEA, 8100004). Plates were inserted into the xCelligence station (ACEA) (37° C., 5% CO₂) and allowed to equilibrate to 37° C. for 1 hour prior to taking background measurement. ARH-77-10B5 cells or Jurkat media were added to the plates (1×10⁴ cells per well) and cultured for 20 h. Each T-cell population was incubated at a final concentration of 10 μM or 0 μM BI224436 in Jurkat media for 1 hour at 37° C. with 5% CO₂. T-cells were added to the plates (1×10⁴ cells per well, 1:1 effector:target ratio) and incubated in the xCelligence station for 24 hours. Data analysis was carried out using xCELLigence Immunotherapy Software (ACEA). The cell index for samples was normalized to the point of T-cell addition, then the normalized cell index for T-cells was divided by the normalized cell index of target cells alone to give the % viable cells at a given timepoint.

### Results

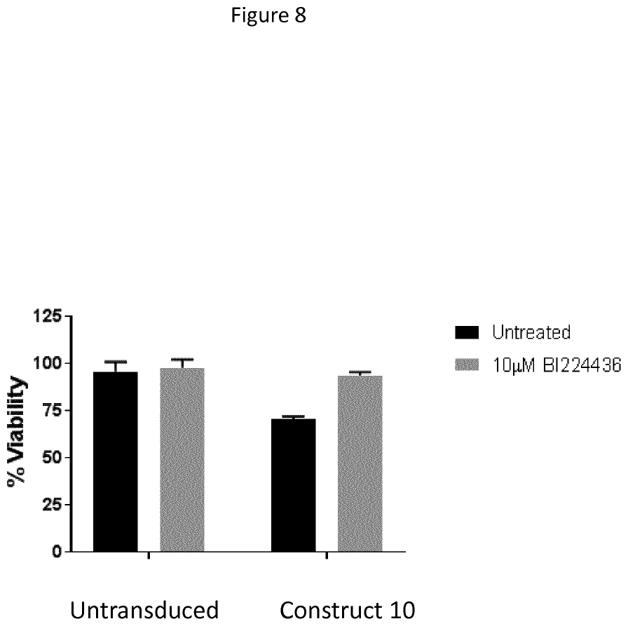

Primary T-cells transduced with the OFF-switch CAR (Construct 10) induce a cytotoxic response against BCMA-positive cells (ARH-77-10B5) (FIG. 8). The response is reduced in the presence of compound BI224436.

### Example 8

### Effect of BI224436 on the Cytotoxicity of Primary T-cells Transduced with the HIV Integrase LEDGF/p75 OFF-Switch CAR or an Equivalent Conventional CAR

The antigen-specific cytotoxicity of the OFF-switch CAR is comparable to an equivalent conventional CAR and BI22436 only attenuates the cytotoxicity of the OFF-switch CAR. Essential CAR components (anti-BCMA scFv, CD8-alpha hinge and transmembrane, 4-1BB, and CD3-zeta) from the OFF-switch CAR were used to generate an equivalent conventional single-chain CAR.

### Materials and Methods

Generation of constructs: Construct 14 was cloned into pG3 vector. Full sequence details of the construct is given below.

CONSTRUCT 14 (SEQ ID NO: 52): this construct is a conventional single chain CAR and uses components from CONSTRUCT 10.

Lentiviral vector production: Method is described in Example 4.

Isolation and transduction of CD4+ and CD8+ T-cells: Peripheral blood mononuclear cells (PBMCs) from the fresh blood of one healthy human donor was isolated by density gradient centrifugation in Accuspin tubes (Sigma) containing 15 mL of Histopaque-1077 (Sigma) and following manufacturer's instructions. CD4+ and CD8+ T cells were isolated using CD4 and CD8 microbeads (Miltenyi Biotec) and the AutoMACS Pro-separator (Miltenyi Biotec). Cells were resuspended at 1×10⁶ cells/mL in TEXMacs media (Miltenyi Biotec) containing 100 units/mL of IL-7 (Sigma), 100 units/mL of IL-15 (Sigma) and TransAct beads (Miltenyi Biotec) and incubated for 48 hours at 37° C. with 5% CO₂.

T-cells were transduced with lentiviral vectors encoding for CONSTRUCT 14 or CONSTRUCT 10. Transduced T-cells and untransduced (UT) T-cells were cultured in TEXMacs media (Miltenyi Biotec) with 100 units/mL of IL-7 (Sigma) and IL-15 (Sigma), and fresh media was added every 3 days. Transduction reactions were prepared to achieve a comparable CAR expression levels between CONSTRUCT 14 and CONSTRUCT 10. 1×10⁵ of each T-cell population was stained with AlexaFluor 647 conjugated BCMA-Fc (generated in-house) to label theanti-BCMA scFv on the CARs. Measurements were made using a Cytoflex S (Beckman Coulter) and data analysed using FlowJo (FlowJo).

K562 BCMA cells (BCMA positive) were cultured in Jurkat media plus 0.8 mg/mL G418 (Gibco) and K562 cells (BCMA negative) were cultured in Jurkat media. Both cell lines were cultured at 37° C. with 5% CO₂. BI224436 compound at a stock concentration of 100 mM in 100% DMSO (Sigma) was diluted in Jurkat media to achieve a stock concentration of 100 μM. K562 cells were stained with Cell Trace Far Red (0.5 μM, ThermoFisher) and K562 BCMA cells were stained with Cell Trace Violet (2.5 μM, ThermoFisher). T-cells were incubated at a final concentration of 10 μM or 0 μM (media alone) BI224436 in Jurkat media for 1 hour at 37° C. with 5% CO2. T-cells were then co-cultured (1×10⁴ K562 or K562 BCMA cells per well, 1:1 or 4:1 effector:target ratio) with K562 or K562 BCMA cells for 48 hours in 10 μM or 0 μM BI224436 Jurkat media at 37° C. with 5% CO₂. After 48 hours, SytoxAADvanced (1 μM, ThermoFisher), Dnase (2.5 mg/ml, ThermoFisher) and EDTA (10 mM, Invitrogen) was added to the wells containing the cells and incubated for 5 min at 25° C. 50 μL of each well was analysed on a Cytoflex S (Beckman Coulter) and data was analysed using FlowJo (FlowJo). The percentage of surviving K562 and K562 BCMA cells was calculated by dividing the number of K562 and K562 BCMA cells in each condition by the number of K562 and K562 BCMA cells found following co-culture with UT T-cells in 0 μM BI224436.

### Results

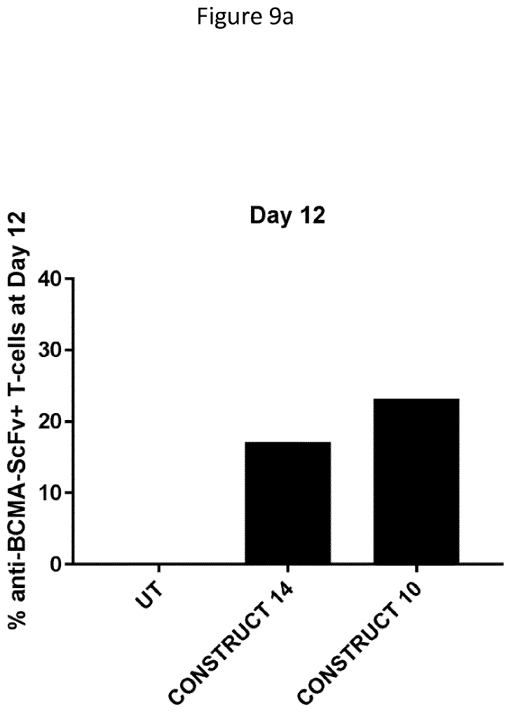

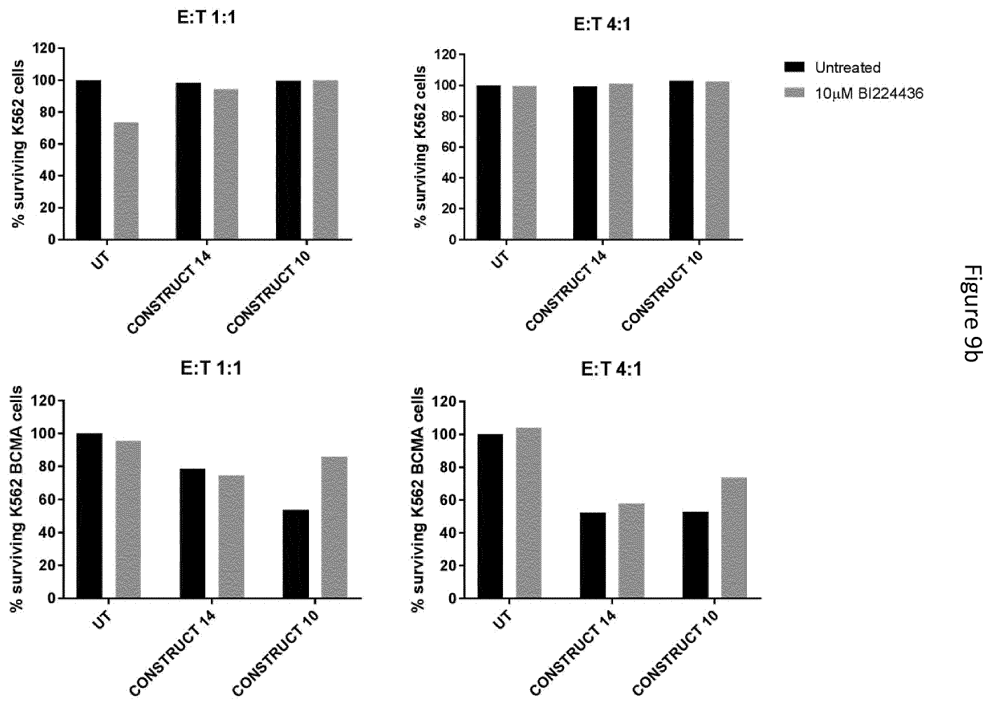

Both primary T-cells transduced with the OFF-switch CAR (Construct 10) and primary T-cells transduced with an equivalent conventional CAR (Construct 14) kill BCMA-positive cells (K562 BCMA) but not BCMA-negative cells (K562 WT) (FIG. 9B). BI224436 does not affect the cytotoxicity of the conventional CAR (Construct 14) but effectively attenuates the cytotoxicity of the OFF-switch CAR (Construct 10).

1. A chimeric antigen receptor (CAR) suitable for the treatment of human subjects, comprising:
(i) a signalling chain comprising a transmembrane domain and a signalling domain;
(ii) a non-signalling chain comprising a target binding domain and a transmembrane domain;
wherein, one of said chains further comprises a HIV Integrase catalytic core domain (CCD) or a functional fragment or variant thereof, and the other chain further comprises a LEDGF/p75 integrase binding domain (IBD), or a functional fragment or variant thereof.

2. The CAR according to claim 1, wherein the signalling chain further comprises a co-stimulatory domain.

3. The CAR according to claim 1, wherein the non-signalling chain further comprises a co-stimulatory domain.

4. The CAR according to claim 1, wherein the non-signalling chain comprises domains in the following order: a target binding domain; a transmembrane domain; a costimulatory domain; and an HIV Integrase CCD or LEDGF/p75 IBD (Arrangement A) and the signalling chain comprises domains in the following order: a transmembrane domain; a costimulatory domain; an HIV Integrase CCD or LEDGF/p75 IBD; and a signalling domain (Arrangement I).

5. The CAR according to claim 1, wherein the non-signalling chain comprises the LEDGF/p75 IBD.

6. The CAR according to claim 1, wherein said LEDGF/p75 IBD functional variants have reduced hydrophobicity at residues 428 and 429.

7. The CAR according to claim 1, wherein said LEDGF/p75 IBD comprises additional amino acid residues from the LEDGF/p75 protein sequence C-terminal to the IBD.

8. The CAR according to claim 7, wherein said LEDGF/p75 IBD comprises an additional 1-45 amino acid residues from the LEDGF/p75 protein sequence C-terminal to the IBD.

9. The CAR of claim 1, wherein the target binding domain comprises an antibody, an antigen binding fragment or a ligand.

10. The CAR of claim 1, wherein the target binding domain binds to a tumour associated antigen.

11. The CAR of claim 10, wherein the tumour associated antigen is selected from: BCMA, carcinoembryonic antigen (CEA), cancer antigen-125, CA19-9, CD5, CD13, CD19, CD20, CD22, CD27, CD30, CD33, CD34, CD45, CD52, CD70, CD117, CD138, CD160, epidermal growth factor receptor (EGFR), folate binding protein, ganglioside G2 (GD2), HER2, mesothelin, MUC-1, neural cell adhesion molecule (NCAM), prostate stem cell antigen (PSCA), prostate-specific membrane antigen (PSMA), prostatic acid phosphatise (PAP), protein melan-A, synaptophysis, six transmembrane epithelial antigen of the prostate I (STEAP1), TARP, Trp-p8, tyrosinase or vimentin.

12. The CAR of claim 1, wherein the transmembrane domain comprises the transmembrane domain of CD8 or CD4.

13. The CAR of claim 1, wherein the signalling domain comprises a CD3zeta signalling domain.

14. The CAR of claim 1, wherein the costimulatory domain is selected from 4-1BB, CD28, CD27, OX40, ICOS, CD30, CD40, PD-1, CD2, CD7, LIGHT, NKG2C, B7-H3 or any combination thereof.

15. (canceled)

16. (canceled)

17. (canceled)

18. A polynucleotide encoding the signalling chain and/or the non-signalling chain of the CAR of claim 1.

19. An expression vector comprising the polynucleotide of claim 16.

20. An immunomodulatory cell comprising the chimeric antigen receptor (CAR) of claim 1.

21. The immunomodulatory cell of claim 20, which is derived from an inflammatory T-lymphocyte, cytotoxic T-lymphocyte, regulatory T-lymphocyte or helper T-lymphocyte.

22. (canceled)

23. A pharmaceutical composition comprising a plurality of immunomodulatory cells according to claim 20.

24. A method of treating and/or preventing a disease, which comprises administering the pharmaceutical composition of claim 23 to a subject.

25. The method of claim 24, which additionally comprises administering an agent which disrupts the interaction between the catalytic core domain (CCD) of the HIV Integrase protein or a functional variant thereof and the human Lens Epithelium-derived Growth factor (LEDGF/p75) protein or functional fragments or variants thereof.

26. The method of claim 25, wherein the agent is administered to the patient before, simultaneously or after the pharmaceutical composition.

27. A method of making an immunomodulatory cell comprising:
(a) providing an immunomodulatory cell;
(b) transducing or transfecting the polynucleotide of claim 18 into said immunomodulatory cell; and
(c) expressing said polynucleotide in the immunomodulatory cell.

28. The method of claim 27, wherein the immunomodulatory cell is allogeneic or autologous.

29. An immunomodulatory cell obtained by the method of claim 27.

30. A method of inhibiting a CAR in a subject which comprises the immunomodulatory cell of claim 20, the method comprising administering to the subject an agent that inhibits the LEDGF/p75-HIV Integrase interaction.


SEQUENCE LISTING (53 sequences)

SEQ ID NO: 1 - protein, 80 aa, Homo Sapiens
SMDSRLQRIHAEIKNSLKIDNLDVNRCIEALDELASLQVTMQQAQKHTEMITTLKKIRRFKVSQVIMEKSTMLYNKFKNM

SEQ ID NO: 2 - protein, 153 aa, Human Immunodeficiency Virus
CSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNFKRKGGIGGYSAGERIVDIIATDI

SEQ ID NO: 3 - protein, 23 aa, Artificial
  Linker
GGGGSGGGGSGGGGSGGGGSDPS

SEQ ID NO: 4 - protein, 23 aa, Artificial
  Linker
GGGGSGGGGSGGGGSGGGGSGGS

SEQ ID NO: 5 - protein, 9 aa, Artificial
  Linker
SDPSGGGGS

SEQ ID NO: 6 - protein, 19 aa, Artificial
  Linker
SDPSGGGGSGGGGSGGGGS

SEQ ID NO: 7 - protein, 4 aa, Artificial
  Linker
GGGS

SEQ ID NO: 8 - protein, 7 aa, Artificial
  Linker
SDPGGGS

SEQ ID NO: 9 - protein, 4 aa, Artificial
  Linker
GGSG

SEQ ID NO: 10 - protein, 37 aa, Homo Sapiens
TRGLDFACDIYIWAPLAGTCGVLLLSLVITLYCNHRN

SEQ ID NO: 11 - protein, 46 aa, Homo Sapiens
FVPVFLPAKPTTTPAPRPPTPAPTIASQPLSLRPEACRPAAGGAVH

SEQ ID NO: 12 - protein, 83 aa, Homo Sapiens
FVPVFLPAKPTTTPAPRPPTPAPTIASQPLSLRPEACRPAAGGAVHTRGLDFACDIYIWAPLAGTCGVLLLSLVITLYCNHRN

SEQ ID NO: 13 - protein, 13 aa, Homo Sapiens
RRRVCKCPRPVVK

SEQ ID NO: 14 - protein, 112 aa, Homo Sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 21 aa, Homo Sapiens
MALPVTALLLPLALLLHAARP

SEQ ID NO: 16 - protein, 42 aa, Homo Sapiens
KRGRKKLLYIFKQPFMRPVQTTQEEDGCSCRFPEEEEGGCEL

SEQ ID NO: 17 - protein, 21 aa, Foot and Mouth Disease Virus
ATNFSLLKQAGDVEENPGPFE

SEQ ID NO: 18 - protein, 22 aa, Artificial
  3x FLAG tag
DYKDHDGDYKDHDIDYKDDDDK

SEQ ID NO: 19 - protein, 227 aa, Artificial
  Anti-BCMA single chain Fv
QVQLVQSGAEVKKPGSSVKVSCKGSGYTFTNYWMHWVRQAPGQGLEWIGATYRGHSDTYYNQKFKGRATLTADTSTSTAYMELSSLRSEDTAVYYCTRGAIYDGYDVLDNWGQGTLVTVDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 20 - protein, 10 aa, Artificial
  Myc tag
EQKLISEEDL

SEQ ID NO: 21 - protein, 851 aa, Artificial
  Construct 1 amino acid sequence
MDYKDHDGDYKDHDIDYKDDDDKGGGSCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNFKRKGGIGGYSAGERIVDIIATDISDPSGGGGSGGGGSGGGGSKRGRKKLLYIFKQPFMRPVQTTQEEDGCSCRFPEEEEGGCELGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGSGATNFSLLKQAGDVEENPGPFEMALPVTALLLPLALLLHAARPQVQLVQSGAEVKKPGSSVKVSCKGSGYTFTNYWMHWVRQAPGQGLEWIGATYRGHSDTYYNQKFKGRATLTADTSTSTAYMELSSLRSEDTAVYYCTRGAIYDGYDVLDNWGQGTLVTVSSGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKGGGGSGGGGSGGGGSGGGGSDPSSMDSRLQRIHAEIKNSLKIDNLDVNRCIEALDELASLQVTMQQAQKHTEMITTLKKIRRFKVSQVIMEKSTMLYNKFKNM

SEQ ID NO: 22 - protein, 841 aa, Artificial
  Construct 2 amino acid sequence
MDYKDHDGDYKDHDIDYKDDDDKGGGSSMDSRLQRIHAEIKNSLKIDNLDVNRCIEALDELASLQVTMQQAQKHTEMITTLKKIRRFKVSQVIMEKSTMLYNKFKNMSDPSGGGGSKRGRKKLLYIFKQPFMRPVQTTQEEDGCSCRFPEEEEGGCELGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGSGATNFSLLKQAGDVEENPGPFEMALPVTALLLPLALLLHAARPQVQLVQSGAEVKKPGSSVKVSCKGSGYTFTNYWMHWVRQAPGQGLEWIGATYRGHSDTYYNQKFKGRATLTADTSTSTAYMELSSLRSEDTAVYYCTRGAIYDGYDVLDNWGQGTLVTVSSGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKGGGGSGGGGSGGGGSGGGGSDPSCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNFKRKGGIGGYSAGERIVDIIATDI

SEQ ID NO: 23 - protein, 902 aa, Artificial
  Construct 3 amino acid sequence
MDYKDHDGDYKDHDIDYKDDDDKGGGSCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNFKRKGGIGGYSAGERIVDIIATDISDPSGGGGSGGGGSGGGGSKRGRKKLLYIFKQPFMRPVQTTQEEDGCSCRFPEEEEGGCELGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGSGATNFSLLKQAGDVEENPGPFEMALPVTALLLPLALLLHAARPQVQLVQSGAEVKKPGSSVKVSCKGSGYTFTNYWMHWVRQAPGQGLEWIGATYRGHSDTYYNQKFKGRATLTADTSTSTAYMELSSLRSEDTAVYYCTRGAIYDGYDVLDNWGQGTLVTVSSGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKGGGGSGGGGSGGGGSGGGGSDPSSMDSRLQRIHAEIKNSLKIDNLDVNRCIEALDELASLQVTMQQAQKHTEMITTLKKIRRFKVSQVIMEKSTMLYNKFKNMSDPSGGGGSKRGRKKLLYIFKQPFMRPVQTTQEEDGCSCRFPEEEEGGCEL

SEQ ID NO: 24 - protein, 902 aa, Artificial
  Construct 4 amino acid sequence
MDYKDHDGDYKDHDIDYKDDDDKGGGSSMDSRLQRIHAEIKNSLKIDNLDVNRCIEALDELASLQVTMQQAQKHTEMITTLKKIRRFKVSQVIMEKSTMLYNKFKNMSDPSGGGGSKRGRKKLLYIFKQPFMRPVQTTQEEDGCSCRFPEEEEGGCELGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGSGATNFSLLKQAGDVEENPGPFEMALPVTALLLPLALLLHAARPQVQLVQSGAEVKKPGSSVKVSCKGSGYTFTNYWMHWVRQAPGQGLEWIGATYRGHSDTYYNQKFKGRATLTADTSTSTAYMELSSLRSEDTAVYYCTRGAIYDGYDVLDNWGQGTLVTVSSGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKGGGGSGGGGSGGGGSGGGGSDPSCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNFKRKGGIGGYSAGERIVDIIATDISDPSGGGGSGGGGSGGGGSKRGRKKLLYIFKQPFMRPVQTTQEEDGCSCRFPEEEEGGCEL

SEQ ID NO: 25 - protein, 942 aa, Artificial
  Construct 5 amino acid sequence
MALPVTALLLPLALLLHAARPDYKDHDGDYKDHDIDYKDDDDKTRGLDFACDIYIWAPLAGTCGVLLLSLVITLYCNHRNKRGRKKLLYIFKQPFMRPVQTTQEEDGCSCRFPEEEEGGCELGGGGSGGGGSGGGGSGGGGSDPSSMDSRLQRIHAEIKNSLKIDNLDVNRCIEALDELASLQVTMQQAQKHTEMITTLKKIRRFKVSQVIMEKSTMLYNKFKNMSDPSGGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGSGATNFSLLKQAGDVEENPGPFEMALPVTALLLPLALLLHAARPQVQLVQSGAEVKKPGSSVKVSCKGSGYTFTNYWMHWVRQAPGQGLEWIGATYRGHSDTYYNQKFKGRATLTADTSTSTAYMELSSLRSEDTAVYYCTRGAIYDGYDVLDNWGQGTLVTVSSGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELGGGGSGGGGSGGGGSGGGGSDPSCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNFKRKGGIGGYSAGERIVDIIATDI

SEQ ID NO: 26 - protein, 942 aa, Artificial
  Construct 6 amino acid sequence
MALPVTALLLPLALLLHAARPDYKDHDGDYKDHDIDYKDDDDKTRGLDFACDIYIWAPLAGTCGVLLLSLVITLYCNHRNKRGRKKLLYIFKQPFMRPVQTTQEEDGCSCRFPEEEEGGCELGGGGSGGGGSGGGGSGGGGSDPSSMDSRLQRIHAEIKNSLKIDNLDVNRCIEALDELASLQVTMQQAQKHTEMITTLKKIRRFKVSQVIMEKSTMLYNKFKNMSDPSGGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGSGATNFSLLKQAGDVEENPGPFEMALPVTALLLPLALLLHAARPQVQLVQSGAEVKKPGSSVKVSCKGSGYTFTNYWMHWVRQAPGQGLEWIGATYRGHSDTYYNQKFKGRATLTADTSTSTAYMELSSLRSEDTAVYYCTRGAIYDGYDVLDNWGQGTLVTVSSGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELGGGGSGGGGSGGGGSGGGGSDPSCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNKKRKGGIGGYSAGERIVDIIATDI

SEQ ID NO: 27 - protein, 958 aa, Artificial
  Construct 7 amino acid sequence
MALPVTALLLPLALLLHAARPQVQLVQSGAEVKKPGSSVKVSCKGSGYTFTNYWMHWVRQAPGQGLEWIGATYRGHSDTYYNQKFKGRATLTADTSTSTAYMELSSLRSEDTAVYYCTRGAIYDGYDVLDNWGQGTLVTVSSGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELGGGGSGGGGSGGGGSGGGGSDPSSMDSRLQRIHAEIKNSLKIDNLDVNRCIEALDELASLQVTMQQAQKHTEMITTLKKIRRFKVSQVIMEKSTMLYNKFKNMSDPGGGSGSGATNFSLLKQAGDVEENPGPFEMALPVTALLLPLALLLHAARPDYKDHDGDYKDHDIDYKDDDDKTRGLDFACDIYIWAPLAGTCGVLLLSLVITLYCNHRNKRGRKKLLYIFKQPFMRPVQTTQEEDGCSCRFPEEEEGGCELGGGGSGGGGSGGGGSGGGGSGGSCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNKKRKGGIGGYSAGERIVDIIATDISDPSGGGGSGGGGSGGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 962 aa, Artificial
  Construct 8 amino acid sequence
MALPVTALLLPLALLLHAARPQVQLVQSGAEVKKPGSSVKVSCKGSGYTFTNYWMHWVRQAPGQGLEWIGATYRGHSDTYYNQKFKGRATLTADTSTSTAYMELSSLRSEDTAVYYCTRGAIYDGYDVLDNWGQGTLVTVSSGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELGGGGSGGGGSGGGGSGGGGSDPSSMDSRLQRIHAEIKNSLKIDNLDVNRCIEALDELASLQVTMQQAQKHTEMITTLKKIRRFKVSQVIMEKSTMLYNKFKNMFLVGSDPGGGSGSGATNFSLLKQAGDVEENPGPFEMALPVTALLLPLALLLHAARPDYKDHDGDYKDHDIDYKDDDDKTRGLDFACDIYIWAPLAGTCGVLLLSLVITLYCNHRNKRGRKKLLYIFKQPFMRPVQTTQEEDGCSCRFPEEEEGGCELGGGGSGGGGSGGGGSGGGGSGGSCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNKKRKGGIGGYSAGERIVDIIATDISDPSGGGGSGGGGSGGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 974 aa, Artificial
  Construct 9 amino acid sequence
MALPVTALLLPLALLLHAARPQVQLVQSGAEVKKPGSSVKVSCKGSGYTFTNYWMHWVRQAPGQGLEWIGATYRGHSDTYYNQKFKGRATLTADTSTSTAYMELSSLRSEDTAVYYCTRGAIYDGYDVLDNWGQGTLVTVSSGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELGGGGSGGGGSGGGGSGGGGSDPSSMDSRLQRIHAEIKNSLKIDNLDVNRCIEALDELASLQVTMQQAQKHTEMITTLKKIRRFKVSQVIMEKSTMLYNKFKNMFLVGEGDSVITQVLNKSDPGGGSGSGATNFSLLKQAGDVEENPGPFEMALPVTALLLPLALLLHAARPDYKDHDGDYKDHDIDYKDDDDKTRGLDFACDIYIWAPLAGTCGVLLLSLVITLYCNHRNKRGRKKLLYIFKQPFMRPVQTTQEEDGCSCRFPEEEEGGCELGGGGSGGGGSGGGGSGGGGSGGSCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNKKRKGGIGGYSAGERIVDIIATDISDPSGGGGSGGGGSGGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 1003 aa, Artificial
  Construct 10 amino acid sequence
MALPVTALLLPLALLLHAARPQVQLVQSGAEVKKPGSSVKVSCKGSGYTFTNYWMHWVRQAPGQGLEWIGATYRGHSDTYYNQKFKGRATLTADTSTSTAYMELSSLRSEDTAVYYCTRGAIYDGYDVLDNWGQGTLVTVSSGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELGGGGSGGGGSGGGGSGGGGSDPSSMDSRLQRIHAEIKNSLKIDNLDVNRCIEALDELASLQVTMQQAQKHTEMITTLKKIRRFKVSQVIMEKSTMLYNKFKNMFLVGEGDSVITQVLNKSLAEQRQHEEANKTKDQGKKGPNKKLEKESDPGGGSGSGATNFSLLKQAGDVEENPGPFEMALPVTALLLPLALLLHAARPDYKDHDGDYKDHDIDYKDDDDKTRGLDFACDIYIWAPLAGTCGVLLLSLVITLYCNHRNKRGRKKLLYIFKQPFMRPVQTTQEEDGCSCRFPEEEEGGCELGGGGSGGGGSGGGGSGGGGSGGSCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNKKRKGGIGGYSAGERIVDIIATDISDPSGGGGSGGGGSGGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 962 aa, Artificial
  Construct 11 amino acid sequence
MALPVTALLLPLALLLHAARPQVQLVQSGAEVKKPGSSVKVSCKGSGYTFTNYWMHWVRQAPGQGLEWIGATYRGHSDTYYNQKFKGRATLTADTSTSTAYMELSSLRSEDTAVYYCTRGAIYDGYDVLDNWGQGTLVTVSSGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELGGGGSGGGGSGGGGSGGGGSDPSSMDSRLQRIHAEIKNSLKIDNLDVNRCIEALDELASLQVTMQQAQKHTEMITTLKKIRRFKVSQVIMEKSTMLYNKFKNMFQRGSDPGGGSGSGATNFSLLKQAGDVEENPGPFEMALPVTALLLPLALLLHAARPDYKDHDGDYKDHDIDYKDDDDKTRGLDFACDIYIWAPLAGTCGVLLLSLVITLYCNHRNKRGRKKLLYIFKQPFMRPVQTTQEEDGCSCRFPEEEEGGCELGGGGSGGGGSGGGGSGGGGSGGSCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNKKRKGGIGGYSAGERIVDIIATDISDPSGGGGSGGGGSGGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 962 aa, Artificial
  Construct 12 amino acid sequence
MALPVTALLLPLALLLHAARPQVQLVQSGAEVKKPGSSVKVSCKGSGYTFTNYWMHWVRQAPGQGLEWIGATYRGHSDTYYNQKFKGRATLTADTSTSTAYMELSSLRSEDTAVYYCTRGAIYDGYDVLDNWGQGTLVTVSSGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELGGGGSGGGGSGGGGSGGGGSDPSSMDSRLQRIHAEIKNSLKIDNLDVNRCIEALDELASLQVTMQQAQKHTEMITTLKKIRRFKVSQVIMEKSTMLYNKFKNMFQQGSDPGGGSGSGATNFSLLKQAGDVEENPGPFEMALPVTALLLPLALLLHAARPDYKDHDGDYKDHDIDYKDDDDKTRGLDFACDIYIWAPLAGTCGVLLLSLVITLYCNHRNKRGRKKLLYIFKQPFMRPVQTTQEEDGCSCRFPEEEEGGCELGGGGSGGGGSGGGGSGGGGSGGSCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNKKRKGGIGGYSAGERIVDIIATDISDPSGGGGSGGGGSGGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 991 aa, Artificial
  Construct 13 amino acid sequence
MALPVTALLLPLALLLHAARPQVQLVQSGAEVKKPGSSVKVSCKGSGYTFTNYWMHWVRQAPGQGLEWIGATYRGHSDTYYNQKFKGRATLTADTSTSTAYMELSSLRSEDTAVYYCTRGAIYDGYDVLDNWGQGTLVTVSSGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELGGGGSGGGGSGGGGSGGGGSDPSSMDSRLQRIHAEIKNSLKIDNLDVNRCIEALDELASLQVTMQQAQKHTEMITTLKKIRRFKVSQVIMEKSTMLYNKFKNMFLVGEGDSVITQVLNKSLAEQRQHEEANKTKDQGKKGPNKKLEKESDPGGGSGSGATNFSLLKQAGDVEENPGPFEMALPVTALLLPLALLLHAARPEQKLISEEDLTRGLDFACDIYIWAPLAGTCGVLLLSLVITLYCNHRNKRGRKKLLYIFKQPFMRPVQTTQEEDGCSCRFPEEEEGGCELGGGGSGGGGSGGGGSGGGGSGGSCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNKKRKGGIGGYSAGERIVDIIATDISDPSGGGGSGGGGSGGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - DNA, 2553 bp, Artificial
  Construct 1 DNA sequence
atggattacaaggaccacgacggcgactacaaagaccacgacatcgactacaaagatgacgacgataagggcggcggcagctgcagccccggcatctggcagctcgattgtacccacctggagggcaaagtgatcctggtcgccgtgcacgtcgccagcggctacatcgaggccgaagtgattcccgcagagaccggccaggagaccgcctacttcctgctcaagctggccggaaggtggcccgtgaagactgtgcacaccgacaacggcagcaacttcaccagcaccaccgtgaaggccgcctgctggtgggccggcatcaaacaggagttcgggatcccctacaacccccagagccagggcgtgatcgagagcatgaacaaggagctgaagaagatcatcgggcaggtgagggaccaggccgagcacctgaagaccgccgtgcagatggccgtgttcatccacaacttcaagaggaagggcggcatcggcggctatagcgccggcgagaggatcgtggacatcatcgccaccgacattagcgaccccagcggaggcggcggcagcggaggcggcggcagcggcggaggcggcagcaagaggggccggaagaagctgctgtacatcttcaagcagcccttcatgaggcccgtgcagaccacacaggaggaagacggctgcagctgcaggtttcccgaggaggaggaaggcggctgcgagctgggcggcggaagcagggtgaagttcagcaggagcgccgacgcccccgcctaccagcagggccagaaccagctgtacaacgagctgaacctgggcaggagggaggagtacgacgtgctggacaaaaggaggggcagggacccagagatgggaggcaagcccagaaggaagaatccccaggaggggctgtacaacgagctgcagaaagacaagatggccgaggcctacagcgagatcggcatgaagggcgagaggaggaggggcaagggccacgacggcctgtaccagggcctgagcaccgctaccaaggacacctacgacgcactgcacatgcaggctctgccccccagaggcggctccggcgccaccaacttctctctcctgaagcaggccggcgacgtggaggagaaccctggccccttcgaaatggccctccccgtgaccgccctgctgctgcccctggcactgctgctccacgccgctaggccacaggtgcagctggtccagagcggcgccgaggtcaagaagcccgggagctccgtgaaggtgagctgcaagggcagcggctacaccttcaccaactactggatgcactgggtgaggcaggctcccggccagggcctggagtggatcggcgccacctacaggggccactccgacacctactacaaccagaagttcaagggcagggccaccctgaccgccgacaccagcaccagcaccgcctacatggaactgagcagcctgaggagcgaggacactgccgtgtactactgcaccaggggcgcaatctacgacggctacgacgtcctggacaactggggccagggcaccctggtgaccgtgtctagcggaggcggaggcagcggcggcggaggatcaggcggcggaggaagcggcggcggcggcagcgacatccagatgactcagagcccctcaagcctgtctgccagcgtgggcgatagggtgaccatcacctgcagcgccagccaggacatcagcaactatctgaactggtaccagcagaagcccggaaaggcccccaagctgctgatctactacactagcaacctgcacagcggcgtgcccagcaggttctcaggaagcggcagcggcaccgactttaccctgaccatcagcagcctgcagcccgaggacttcgccacctactactgccagcagtacaggaagctgccctggaccttcggccagggcaccaaactggagatcaagaggttcgtgcccgtgttcctgcccgccaaacctaccacaacccccgcccccagaccccccacccccgcccccaccattgccagccagcccctgagcctgaggcccgaggcctgtagacccgccgccgggggagctgtgcacaccaggggcctggacttcgcctgcgatatctacatctgggcccccctggctggaacctgcggcgtgctgctgctcagcctggtgatcacactgtactgcaaccacaggaaccggaggagggtgtgcaagtgccccaggcctgtggtgaagggcggaggcggcagcggcggcggcggcagcggaggcgggggcagcggagggggcggcagcgaccccagcagcatggacagcaggctgcagaggatccacgccgagatcaagaacagcctgaagatcgacaacctggacgtgaacaggtgcatcgaagccctggacgagctggccagcctgcaggtgaccatgcagcaggcccagaagcacaccgagatgatcaccaccctgaagaagatcaggaggttcaaggtgagccaggtgattatggagaagagcaccatgctctataataagttcaagaacatg

SEQ ID NO: 35 - DNA, 2523 bp, Artificial
  Construct 2 DNA sequence
atggactacaaggaccacgacggcgactacaaagaccacgacatcgactacaaagatgacgacgataagggcggcggcagctctatggattccaggctccagaggatccacgccgagatcaagaacagcctgaagatcgacaacctggacgtcaacaggtgcattgaagccctcgacgagctggccagcctgcaggtgaccatgcagcaggcccagaagcacactgagatgatcaccaccctgaagaagatcaggcggttcaaggtgagccaggtgatcatggagaagagcaccatgctgtacaacaagttcaagaacatgtcagaccccagcggcggcggcggcagcaagaggggcaggaagaagctgctgtacatcttcaagcagcccttcatgagacccgtgcagaccacccaggaggaggacggctgcagctgcagattccctgaagaggaggagggcggctgcgaactgggcggcggaagcagggtgaagttcagcaggagcgccgacgcccccgcctaccagcagggccagaaccagctgtacaacgagctgaacctgggccgcagggaggagtacgacgtgctggacaaaaggaggggcagggaccccgagatgggcggcaagcccaggaggaaaaacccccaggagggactgtacaacgagctgcagaaggacaagatggccgaggcctacagcgaaatcggcatgaagggcgagagaaggaggggcaagggccacgacggcctgtatcagggcctgagcaccgccaccaaggatacctacgacgcactgcacatgcaggctctgccccccagggggggcagcggggccaccaactttagcctgctgaaacaggccggcgacgtggaggaaaatcccggacccttcgaaatggctctgcccgtgaccgccctgctgctgcccctcgctctgctcctgcacgcagccaggccccaggtgcagctggtccagagcggcgccgaggtgaagaagcccggcagcagcgtgaaggtgagctgcaagggcagcggctacaccttcaccaactactggatgcactgggtgaggcaggctcccggccagggcctggagtggatcggcgccacctacaggggccatagcgacacctactacaaccagaagttcaagggaagggccacactgaccgccgacacttcaaccagcaccgcctacatggaactgagcagcctgaggagcgaggacaccgccgtgtactactgcaccaggggcgccatctacgacggctacgacgtgctggacaactggggccagggcaccctggtgactgtgagcagcggcggaggaggcagcggaggcggcggcagcggcggaggcggcagcggcggcggcggcagcgacattcagatgacccagagccccagcagcctgagcgccagcgtgggcgacagggtgaccatcacctgcagcgccagccaggacatcagcaactacctgaactggtatcagcagaagcccggcaaggcccccaagctgctgatctactacacctcaaacctgcactccggcgtcccctctaggttcagcggcagcggcagcggcaccgactttaccctcaccatcagcagcctgcagcccgaagacttcgccacctactactgccagcagtacaggaagctgccctggaccttcggccagggcaccaagctggaaatcaagaggttcgtgcccgtgttcctgcccgccaaacccaccaccactcctgcccccagaccccccacccccgcccccacaattgcctctcagcccctgagcctgaggccagaggcctgcagacctgccgccggcggcgccgtgcacaccaggggcctggacttcgcctgcgacatctacatctgggctcccctggccggcacctgcggggtgctgctgctgagcctggtgatcaccctctactgcaatcacaggaacaggaggcgcgtgtgcaagtgccccaggcccgtggtgaaaggcggaggcgggagcggcggaggaggcagcggaggcggcggctccggaggcggcggcagcgatcccagctgtagcccaggcatctggcagctcgattgcacccacctggagggcaaagtgatcctggtggccgtgcacgtggccagcggctacatcgaggcagaggtgatccccgccgagaccggccaggagaccgcctactttctgctgaagctggccggcaggtggcccgtcaaaaccgtgcacaccgacaacggcagcaacttcaccagcaccaccgtgaaggcagcctgctggtgggccggcatcaaacaggagttcggcatcccctacaacccccagagccagggagtgatcgagagcatgaacaaggagctgaagaagattatcggccaggtccgggaccaggccgagcacctgaagaccgccgtgcagatggccgtgttcatccacaacttcaagaggaaggggggcatcggcggctatagcgccggcgagaggatcgtggacatcatcgccacagacatc

SEQ ID NO: 36 - DNA, 2706 bp, Artificial
  Construct 3 DNA sequence
atggactacaaggatcacgacggcgactataaggaccacgacatcgactacaaagacgacgacgacaagggcggcggctcctgtagccctggcatctggcagctcgactgcacccacctggagggcaaagtgatcctggtggccgtgcacgtggccagcggctacatcgaggctgaggtgattcccgccgaaaccggccaggagacagcctacttcctgctgaagctggccggcaggtggccagtgaagaccgtgcacaccgacaacggcagcaacttcaccagcaccaccgtgaaggccgcctgctggtgggctggcatcaagcaggagttcgggatcccctacaacccccagagccagggcgtgatcgagagcatgaacaaggagctgaagaagatcatcggccaggtccgggatcaggccgagcacctgaagaccgccgtgcagatggccgtgttcatccacaacttcaagaggaagggcggcatcggcggctatagcgccggcgagaggatcgtggacatcatcgcaaccgatatcagcgaccccagcggaggcggcggcagcggaggcggcggatcaggaggcggcggcagcaaaaggggccgcaagaagctgctgtacatcttcaagcagcccttcatgaggcccgtgcagaccacccaggaggaggacggctgcagctgcaggttccctgaggaagaagagggaggctgcgagctgggaggcggcagcagggtgaagttctctaggagcgccgacgcccccgcctaccagcagggccagaaccagctgtacaacgagctgaacctgggcaggagggaggagtacgacgtgctggacaagaggaggggcagggatcccgagatgggaggcaaacccaggaggaagaacccccaggagggcctgtacaacgagctgcagaaggacaagatggccgaggcctacagcgagatcggcatgaaaggcgagaggaggaggggcaaaggccacgacgggctgtatcagggcctgagcaccgccaccaaggacacctacgacgccctgcacatgcaggctctgccccccaggggcggcagcggcgccaccaacttcagcctgctcaaacaggccggcgacgtggaagagaaccccggccccttcgaaatggccctgcctgtgaccgccctgctgctccccctggctctgctcctgcacgccgcaaggccccaggtccagctggtgcagagcggcgccgaggtgaagaaacccggcagcagcgtgaaagtgagctgcaagggcagcggctacaccttcaccaactactggatgcattgggtgaggcaggcccccgggcagggactggagtggatcggcgccacctacaggggccacagcgacacctactacaaccagaagttcaagggcagggccaccctgaccgccgacaccagcaccagcactgcctacatggagctgagcagcctgaggagcgaggacaccgccgtgtactattgcaccaggggcgcaatctacgacggctacgacgtcctggacaactggggccagggaaccctggtgacagtgagcagcggcggcggcggatcaggcggcggcggctctggcggcggcggaagcggcggcggaggcagcgatatccagatgacccagtctcccagcagcctgagcgccagcgtgggcgacagggtgaccatcacctgcagcgccagccaggacattagcaactacctgaactggtaccagcagaagcccggcaaggcccccaagctgctgatctactacacctctaacctccacagcggcgtgccaagcaggtttagcggctccggaagcggcaccgatttcaccctcaccatcagcagcctgcagcccgaggacttcgccacctactactgccagcagtacaggaagctgccctggaccttcggccagggcaccaagctggagatcaagaggttcgtgcccgtgttcctgcccgccaagcccaccacaacccccgcccccaggccccccacccccgcccccaccattgcctctcagcccctgtctctgaggcccgaggcctgcaggcctgccgccggcggagccgtgcacaccaggggcctggacttcgcctgcgacatctacatctgggcccccctggctgggacttgcggcgtgctgctgctgagcctggtgatcaccctgtactgcaaccacagaaataggaggagggtgtgcaagtgccccaggcccgtggtgaaaggaggcggcggcagcggcggcggcggcagcggcggcggaggcagcggcggcggaggcagcgaccccagcagcatggacagcaggctgcagaggatccacgccgagatcaagaactcactgaagatcgacaacctggacgtgaaccgctgcatcgaagccctggacgagctggcctccctgcaggtgaccatgcagcaggcccagaagcacaccgagatgatcaccaccctgaagaagatcaggaggttcaaggtgagccaggtgatcatggagaagagcaccatgctctacaacaagttcaagaacatgagcgaccccagcggcggaggcggcagcaagaggggcaggaagaaactgctgtatatcttcaagcagccctttatgcggcccgtccagaccacccaggaggaggacgggtgcagctgcaggttccccgaagaggaggagggcgggtgtgagctg

SEQ ID NO: 37 - DNA, 2709 bp, Artificial
  Construct 4 DNA sequence
atggactacaaggaccacgacggcgactataaggaccacgacatcgactacaaagacgacgacgataagggcggcggcagcagcatggacagcaggctccagaggatccacgccgagatcaagaacagcctgaagatcgacaacctggacgtcaacaggtgcatcgaggccctggacgagctcgctagcctgcaggtgaccatgcagcaggcccagaagcacaccgagatgatcaccaccctcaagaaaatcaggcggttcaaggtgagccaggtgatcatggagaagagcaccatgctgtacaacaagttcaagaacatgagcgatcccagcggcggcggcggaagcaaaaggggcaggaagaagctgctgtacatcttcaagcagcccttcatgagacccgtgcagaccacccaggaggaggacggctgctcttgcaggttccccgaagaggaggaaggaggctgtgagctgggaggcggcagcagggtgaagttcagcaggagcgccgacgcccccgcctaccagcagggccagaaccagctgtacaacgagctgaacctgggaaggagggaagagtacgacgtgctggacaaaaggaggggcagggaccccgagatgggcggcaagcctaggaggaagaacccccaggagggcctgtacaacgagctgcagaaggacaagatggccgaggcctacagcgagattgggatgaagggggagaggaggaggggcaagggccacgacggcctgtaccagggcctgagcaccgccaccaaggacacttacgacgccctgcacatgcaggccctgccccccagaggcggcagcggcgcaaccaacttttccctgctgaagcaggccggcgacgtggaggagaatcccggacccttcgaaatggccctgcccgtgactgccctgctgctgcccctggccctgctgctgcacgcagccaggccccaggtccagctggtgcagagcggcgccgaagtgaagaagcccggcagcagcgtgaaggtgagctgcaagggcagcggctacaccttcaccaactactggatgcactgggtgaggcaggcccctggccagggcctggagtggatcggcgccacctacaggggccacagcgacacctactacaaccagaagttcaagggcagggccaccctgaccgccgacaccagcacaagcaccgcctacatggaactgagcagcctgaggagcgaggacaccgccgtgtactactgcacccggggcgccatctacgacggctacgacgtgctggacaactggggccagggcaccctggtgaccgtgtctagcggcggcggaggcagcgggggcggcggaagcggaggcggcggctctggcggcggcggaagcgatatccagatgacccagagccccagcagcctgagcgctagcgtgggcgacagggtgaccatcacctgcagcgccagccaggacatcagcaactacctgaactggtaccagcagaagcccggcaaggcccccaagctgctgatctactacacctcaaacctgcactcaggcgtgcccagcaggtttagcggcagcggctccggcaccgacttcaccctgaccatcagcagcctgcagcccgaggacttcgccacctattactgccagcagtacaggaagctgccctggaccttcggccagggcaccaagctggagatcaagaggttcgtgcccgtgttcctccccgccaaacccaccactacacccgccccaaggccacctactcccgcccccacaattgcctcccagcccctgtcactgaggcccgaggcctgcagacccgccgcaggcggagccgtgcacaccaggggcctggacttcgcctgcgacatctacatctgggcccccctggccggcacctgcggcgtgctgctgctcagcctggtgatcaccctctactgcaaccacaggaaccggaggagggtgtgcaagtgccccaggcccgtggtgaaaggcgggggcggcagcggcggcggcggcagcggcggcggcggcagcggcggcggaggcagcgaccccagctgcagccccggcatctggcagctggactgtacccacctggagggcaaagtgatcctcgtcgccgtgcacgtcgccagcggctacatcgaggccgaagtgattcccgccgagaccggccaggagaccgcctacttcctgctgaaactggccggcaggtggcccgtgaagaccgtgcacaccgacaacggcagcaacttcaccagcactaccgtgaaggccgcctgttggtgggccggcatcaagcaggagttcggcatcccctacaacccccagagccagggcgtgatcgagagcatgaataaggagctcaagaagatcatcggccaggtcagggaccaggccgagcacctgaagaccgccgtgcagatggctgtgttcatccacaacttcaagcgcaaggggggcatcggcggctatagcgccggcgagaggatcgtggacatcatcgctaccgacatcagcgatccctctggcggcggcggaagcggcggcggcggcagcggcggcggcggaagcaagaggggcaggaagaagctgctgtacatcttcaagcagcccttcatgcgccccgtgcagaccacccaggaggaggacgggtgcagctgcaggtttcccgaggaggaggagggcggctgcgagctgtga

SEQ ID NO: 38 - DNA, 2826 bp, Artificial
  Construct 5 DNA sequence
atggccctgcccgtcaccgccctgctgctccccctggccctgctgctgcacgccgccagacccgactacaaggaccacgacggcgactacaaagaccacgacatcgactacaaagatgacgacgacaagaccaggggcctcgacttcgcctgcgacatctacatttgggcccccctggccggcacctgtggcgtgctcctgctgagcctggtgatcaccctgtactgcaaccacaggaacaagaggggcaggaagaagctgctgtacatcttcaagcagcccttcatgaggcccgtgcagactacccaggaggaggacggctgcagctgcaggttccccgaggaagaggaaggcggctgcgagctgggcgggggcggaagcggcggcggcggctcaggaggcggcggaagcggcggcggcggcagcgatcccagcagcatggatagcaggctgcagaggatccacgccgagatcaagaactccctgaagatcgacaacctggacgtgaacaggtgcatcgaggccctggacgagctggccagcctgcaggtgaccatgcagcaggctcagaagcacaccgagatgatcaccaccctgaagaagatccggaggttcaaggtgagccaggtgatcatggagaagagcaccatgctgtacaacaagttcaagaatatgagcgaccccagcggaggcggcggcagcagggtgaaattctctaggagcgccgacgcccccgcctaccagcagggccagaaccagctgtacaacgagctgaacctgggccgccgggaggagtacgacgtgctggataagaggaggggcagggaccccgagatgggcggcaaacccaggaggaagaaccctcaggagggcctgtacaacgagctgcagaaggacaagatggccgaggcctacagcgagatcggcatgaaaggcgagaggaggaggggcaagggccacgacggcctgtatcagggcctgagcacagccaccaaggacacctacgacgccctgcacatgcaggccctgcccccaagggggggcagcggcgccaccaactttagcctgctcaagcaggcaggcgacgtggaggagaatcccggccccttcgaaatggccctgcctgtgaccgcactgctgctgcccctggccctgctgctgcacgctgccaggccccaggtgcagctggtgcagagcggcgcagaggtgaagaagcccggcagcagcgtgaaggtgagctgcaaaggcagcggctacacctttaccaactactggatgcactgggtcaggcaggctcccggacagggcctggagtggatcggcgccacctacaggggccactcagacacctactacaaccagaagttcaagggcagggccaccctgaccgccgacaccagcaccagcaccgcctacatggaactgagcagcctgaggagcgaggacaccgccgtgtactactgcaccaggggcgccatctacgacggctacgacgtgctcgacaactggggccagggaacactcgtgaccgtgagcagcggcggcggcggaagcggaggcggcggaagcggcggcggcggctctggcggcgggggctccgacattcagatgacccagagccccagcagcctgagcgccagcgtgggcgacagggtgaccatcacctgcagcgccagccaggacatcagcaactacctcaactggtaccagcagaagcccggcaaggcccccaagctgctgatctactacacctccaatctccacagcggggtgcccagcaggtttagcggaagcgggagcggcaccgatttcaccctgaccatcagcagcctgcagcccgaggacttcgccacctattactgccagcagtacaggaaactgccctggaccttcggccagggcaccaagctggagattaagaggttcgtgcccgtgttcctgcccgccaaacccaccacaacccctgcccccagaccccccacccccgctcccactatcgcctcacagcccctgtctctgaggcccgaagcctgtaggcccgctgccggcggcgccgtgcacaccagaggcctggacttcgcctgcgacatctatatctgggcccccctggccggcacttgcggcgtcctgctgctcagcctggtgatcaccctgtactgcaaccaccggaacaagaggggcaggaagaagctcctgtacatcttcaagcagcccttcatgaggcccgtccagaccacccaggaggaggacggctgcagctgcaggttcccagaggaagaagagggcggctgcgagctgggcggcggcggaagcggcggcggcggaagcggcggcggcggcagcggcggcggcggcagcgatcccagctgcagccccggcatctggcagctggactgcacccacctggagggcaaggtcatcctggtggccgtgcacgtggccagcggctatatcgaggccgaggtgatccccgccgagaccggacaggagaccgcctacttcctgctgaagctggccggcaggtggcccgtgaagaccgtgcacaccgacaacggcagcaacttcaccagcactaccgtgaaggccgcctgctggtgggccggcatcaagcaggagttcggcatcccctacaacccccagagccagggcgtgatcgagtccatgaacaaggagctgaagaagatcatcggccaggtgagggaccaggccgagcacctgaagaccgccgtgcagatggccgtgttcatccacaatttcaagaggaagggcggcattggaggctacagcgccggcgagaggatcgtggacatcatcgccaccgacatc

SEQ ID NO: 39 - DNA, 2826 bp, Artificial
  Construct 6 DNA sequence
atggccctgcccgtcaccgccctgctgctccccctggccctgctgctgcacgccgccagacccgactacaaggaccacgacggcgactacaaagaccacgacatcgactacaaagatgacgacgacaagaccaggggcctcgacttcgcctgcgacatctacatttgggcccccctggccggcacctgtggcgtgctcctgctgagcctggtgatcaccctgtactgcaaccacaggaacaagaggggcaggaagaagctgctgtacatcttcaagcagcccttcatgaggcccgtgcagactacccaggaggaggacggctgcagctgcaggttccccgaggaagaggaaggcggctgcgagctgggcgggggcggaagcggcggcggcggctcaggaggcggcggaagcggcggcggcggcagcgatcccagcagcatggatagcaggctgcagaggatccacgccgagatcaagaactccctgaagatcgacaacctggacgtgaacaggtgcatcgaggccctggacgagctggccagcctgcaggtgaccatgcagcaggctcagaagcacaccgagatgatcaccaccctgaagaagatccggaggttcaaggtgagccaggtgatcatggagaagagcaccatgctgtacaacaagttcaagaatatgagcgaccccagcggaggcggcggcagcagggtgaaattctctaggagcgccgacgcccccgcctaccagcagggccagaaccagctgtacaacgagctgaacctgggccgccgggaggagtacgacgtgctggataagaggaggggcagggaccccgagatgggcggcaaacccaggaggaagaaccctcaggagggcctgtacaacgagctgcagaaggacaagatggccgaggcctacagcgagatcggcatgaaaggcgagaggaggaggggcaagggccacgacggcctgtatcagggcctgagcacagccaccaaggacacctacgacgccctgcacatgcaggccctgcccccaagggggggcagcggcgccaccaactttagcctgctcaagcaggcaggcgacgtggaggagaatcccggccccttcgaaatggccctgcctgtgaccgcactgctgctgcccctggccctgctgctgcacgctgccaggccccaggtgcagctggtgcagagcggcgcagaggtgaagaagcccggcagcagcgtgaaggtgagctgcaaaggcagcggctacacctttaccaactactggatgcactgggtcaggcaggctcccggacagggcctggagtggatcggcgccacctacaggggccactcagacacctactacaaccagaagttcaagggcagggccaccctgaccgccgacaccagcaccagcaccgcctacatggaactgagcagcctgaggagcgaggacaccgccgtgtactactgcaccaggggcgccatctacgacggctacgacgtgctcgacaactggggccagggaacactcgtgaccgtgagcagcggcggcggcggaagcggaggcggcggaagcggcggcggcggctctggcggcgggggctccgacattcagatgacccagagccccagcagcctgagcgccagcgtgggcgacagggtgaccatcacctgcagcgccagccaggacatcagcaactacctcaactggtaccagcagaagcccggcaaggcccccaagctgctgatctactacacctccaatctccacagcggggtgcccagcaggtttagcggaagcgggagcggcaccgatttcaccctgaccatcagcagcctgcagcccgaggacttcgccacctattactgccagcagtacaggaaactgccctggaccttcggccagggcaccaagctggagattaagaggttcgtgcccgtgttcctgcccgccaaacccaccacaacccctgcccccagaccccccacccccgctcccactatcgcctcacagcccctgtctctgaggcccgaagcctgtaggcccgctgccggcggcgccgtgcacaccagaggcctggacttcgcctgcgacatctatatctgggcccccctggccggcacttgcggcgtcctgctgctcagcctggtgatcaccctgtactgcaaccaccggaacaagaggggcaggaagaagctcctgtacatcttcaagcagcccttcatgaggcccgtccagaccacccaggaggaggacggctgcagctgcaggttcccagaggaagaagagggcggctgcgagctgggcggcggcggaagcggcggcggcggaagcggcggcggcggcagcggcggcggcggcagcgatcccagctgcagccccggcatctggcagctggactgcacccacctggagggcaaggtcatcctggtggccgtgcacgtggccagcggctatatcgaggccgaggtgatccccgccgagaccggacaggagaccgcctacttcctgctgaagctggccggcaggtggcccgtgaagaccgtgcacaccgacaacggcagcaacttcaccagcactaccgtgaaggccgcctgctggtgggccggcatcaagcaggagttcggcatcccctacaacccccagagccagggcgtgatcgagtccatgaacaaggagctgaagaagatcatcggccaggtgagggaccaggccgagcacctgaagaccgccgtgcagatggccgtgttcatccacaataagaagaggaagggcggcattggaggctacagcgccggcgagaggatcgtggacatcatcgccaccgacatc

SEQ ID NO: 40 - DNA, 2874 bp, Artificial
  Construct 7 DNA sequence
atggctctccccgtgaccgccctgctgctgcccctggccctgctgctgcacgccgccaggccccaggtgcagctggtccagagcggcgccgaggtgaagaagcctggcagcagcgtgaaggtgagctgcaagggcagcggctacaccttcaccaactactggatgcactgggtgaggcaggcccccggccagggcctggagtggattggggccacctacaggggccacagcgacacctactacaaccagaagttcaagggcagggccaccctgaccgccgataccagcaccagcaccgcctacatggaactgagcagcctgaggtccgaggacaccgccgtgtactactgcaccagaggcgccatctacgacggctacgacgtgctcgacaactggggccagggcaccctggtgaccgtgtctagcggcggcggcggcagcggcggcggcggcagcggaggcggcggatcaggcggcggcggcagcgatatccagatgacccagagcccctcctctctgagcgccagcgtgggcgatagggtgaccatcacctgcagcgccagccaggacatcagcaactacctgaactggtaccagcagaagcccggcaaggcccccaagctgctgatctactacaccagcaacctgcacagcggcgtgcccagcaggtttagcggcagcggcagcggcaccgacttcaccctgaccatcagctccctgcagcccgaggacttcgccacctactactgccagcagtacaggaagctgccctggaccttcggccagggcaccaagctggagatcaagaggttcgtgcccgtgttcctgcccgccaaacccacaaccactcccgcccccaggccccctacacccgctcccaccattgccagccagcccctcagcctgaggccagaggcctgtaggcccgctgccggaggcgctgtgcacaccaggggcctggacttcgcctgcgacatctacatctgggcacccctggctggcacctgcggcgtgctgctgctgagcctggtgatcaccctgtactgcaaccacaggaacaagaggggcaggaagaagctgctgtacatcttcaagcagcccttcatgaggcccgtgcagaccacccaggaggaggacggctgctcttgcaggttccccgaagaggaggagggcggctgcgagctgggaggagggggaagcggaggcggcggctcaggcggcggcggaagcggcggcggcggcagcgaccccagcagcatggacagcaggctgcagaggatccacgccgagatcaagaacagcctcaagatcgacaacctggacgtgaacaggtgcatcgaggccctggacgagctggccagcctgcaggtgaccatgcagcaggcccagaagcacaccgagatgatcaccaccctgaagaagatcaggaggttcaaggtcagccaggtgatcatggagaagagcaccatgctgtacaacaagttcaagaacatgagcgaccccggcggcggcagcggcagcggagccaccaatttcagcctgctgaagcaggccggagacgtggaggagaatcccggccccttcgaaatggccctgcccgtgaccgccctgctcctgcccctggccctgctgctgcacgccgcaaggcccgactataaggaccacgacggcgactacaaggaccacgacatcgactacaaggacgacgacgacaaaaccaggggcctggacttcgcctgcgacatctacatctgggcccccctggccggcacttgcggcgtgctgctgctgagcctcgtgatcaccctgtactgcaaccacaggaacaagcggggcaggaagaagctcctgtacatcttcaagcagcccttcatgaggcccgtgcagacaacccaggaggaggacggctgcagctgcaggttcccagaggaagaggagggcggctgcgagctggggggcggcggcagcggaggcggaggcagcggcggcggcggcagcggcggcggcggcagcggaggcagctgcagccccggcatctggcagctggactgcacccacctggaggggaaggtgattctggtggccgtgcacgtggcaagcggctatatcgaggccgaggtgattcccgccgagaccggccaggagaccgcctattttctgctcaagctggccggccggtggcccgtcaaaaccgtgcacaccgacaacggcagcaacttcaccagcaccaccgtcaaggccgcctgttggtgggccggcatcaagcaggagttcggcatcccctataacccccagagccagggcgtgatcgagagcatgaataaggagctgaagaagatcatcggccaggtgagggaccaggccgagcacctgaagaccgccgtgcagatggccgtgttcatccacaacaagaagaggaaggggggcatcggcggctacagcgccggcgagaggatcgtggatatcatcgccaccgacatctcagatccctcaggcggcggcggcagcggcggcggcggcagcggcggcggcggcagcagggtgaaatttagcaggagcgccgacgcccccgcctaccagcagggccagaaccagctgtacaacgagctgaacctgggccgcagggaggaatacgacgtgctggacaaaaggaggggcagggacccagagatgggcggcaagcccaggaggaagaacccccaggagggcctgtacaacgagctgcagaaggacaagatggccgaggcctacagcgagatcgggatgaaaggcgaaaggaggagaggcaagggccacgacggcctgtaccagggcctgagcaccgccactaaggacacctacgacgccctgcacatgcaggccctgccccccagg

SEQ ID NO: 41 - DNA, 2886 bp, Artificial
  Construct 8 DNA sequence
atggctctccccgtgaccgccctgctgctgcccctggccctgctgctgcacgccgccaggccccaggtgcagctggtccagagcggcgccgaggtgaagaagcctggcagcagcgtgaaggtgagctgcaagggcagcggctacaccttcaccaactactggatgcactgggtgaggcaggcccccggccagggcctggagtggattggggccacctacaggggccacagcgacacctactacaaccagaagttcaagggcagggccaccctgaccgccgataccagcaccagcaccgcctacatggaactgagcagcctgaggtccgaggacaccgccgtgtactactgcaccagaggcgccatctacgacggctacgacgtgctcgacaactggggccagggcaccctggtgaccgtgtctagcggcggcggcggcagcggcggcggcggcagcggaggcggcggatcaggcggcggcggcagcgatatccagatgacccagagcccctcctctctgagcgccagcgtgggcgatagggtgaccatcacctgcagcgccagccaggacatcagcaactacctgaactggtaccagcagaagcccggcaaggcccccaagctgctgatctactacaccagcaacctgcacagcggcgtgcccagcaggtttagcggcagcggcagcggcaccgacttcaccctgaccatcagctccctgcagcccgaggacttcgccacctactactgccagcagtacaggaagctgccctggaccttcggccagggcaccaagctggagatcaagaggttcgtgcccgtgttcctgcccgccaaacccacaaccactcccgcccccaggccccctacacccgctcccaccattgccagccagcccctcagcctgaggccagaggcctgtaggcccgctgccggaggcgctgtgcacaccaggggcctggacttcgcctgcgacatctacatctgggcacccctggctggcacctgcggcgtgctgctgctgagcctggtgatcaccctgtactgcaaccacaggaacaagaggggcaggaagaagctgctgtacatcttcaagcagcccttcatgaggcccgtgcagaccacccaggaggaggacggctgctcttgcaggttccccgaagaggaggagggcggctgcgagctgggaggagggggaagcggaggcggcggctcaggcggcggcggaagcggcggcggcggcagcgaccccagcagcatggacagcaggctgcagaggatccacgccgagatcaagaacagcctcaagatcgacaacctggacgtgaacaggtgcatcgaggccctggacgagctggccagcctgcaggtgaccatgcagcaggcccagaagcacaccgagatgatcaccaccctgaagaagatcaggaggttcaaggtcagccaggtgatcatggagaagagcaccatgctgtacaacaagttcaagaacatgttcctggtcggaagcgaccccggcggcggcagcggcagcggagccaccaatttcagcctgctgaagcaggccggagacgtggaggagaatcccggccccttcgaaatggccctgcccgtgaccgccctgctcctgcccctggccctgctgctgcacgccgcaaggcccgactataaggaccacgacggcgactacaaggaccacgacatcgactacaaggacgacgacgacaaaaccaggggcctggacttcgcctgcgacatctacatctgggcccccctggccggcacttgcggcgtgctgctgctgagcctcgtgatcaccctgtactgcaaccacaggaacaagcggggcaggaagaagctcctgtacatcttcaagcagcccttcatgaggcccgtgcagacaacccaggaggaggacggctgcagctgcaggttcccagaggaagaggagggcggctgcgagctggggggcggcggcagcggaggcggaggcagcggcggcggcggcagcggcggcggcggcagcggaggcagctgcagccccggcatctggcagctggactgcacccacctggaggggaaggtgattctggtggccgtgcacgtggcaagcggctatatcgaggccgaggtgattcccgccgagaccggccaggagaccgcctattttctgctcaagctggccggccggtggcccgtcaaaaccgtgcacaccgacaacggcagcaacttcaccagcaccaccgtcaaggccgcctgttggtgggccggcatcaagcaggagttcggcatcccctataacccccagagccagggcgtgatcgagagcatgaataaggagctgaagaagatcatcggccaggtgagggaccaggccgagcacctgaagaccgccgtgcagatggccgtgttcatccacaacaagaagaggaaggggggcatcggcggctacagcgccggcgagaggatcgtggatatcatcgccaccgacatctcagatccctcaggcggcggcggcagcggcggcggcggcagcggcggcggcggcagcagggtgaaatttagcaggagcgccgacgcccccgcctaccagcagggccagaaccagctgtacaacgagctgaacctgggccgcagggaggaatacgacgtgctggacaaaaggaggggcagggacccagagatgggcggcaagcccaggaggaagaacccccaggagggcctgtacaacgagctgcagaaggacaagatggccgaggcctacagcgagatcgggatgaaaggcgaaaggaggagaggcaagggccacgacggcctgtaccagggcctgagcaccgccactaaggacacctacgacgccctgcacatgcaggccctgccccccagg

SEQ ID NO: 42 - DNA, 2922 bp, Artificial
  Construct 9 DNA sequence
atggctctccccgtgaccgccctgctgctgcccctggccctgctgctgcacgccgccaggccccaggtgcagctggtccagagcggcgccgaggtgaagaagcctggcagcagcgtgaaggtgagctgcaagggcagcggctacaccttcaccaactactggatgcactgggtgaggcaggcccccggccagggcctggagtggattggggccacctacaggggccacagcgacacctactacaaccagaagttcaagggcagggccaccctgaccgccgataccagcaccagcaccgcctacatggaactgagcagcctgaggtccgaggacaccgccgtgtactactgcaccagaggcgccatctacgacggctacgacgtgctcgacaactggggccagggcaccctggtgaccgtgtctagcggcggcggcggcagcggcggcggcggcagcggaggcggcggatcaggcggcggcggcagcgatatccagatgacccagagcccctcctctctgagcgccagcgtgggcgatagggtgaccatcacctgcagcgccagccaggacatcagcaactacctgaactggtaccagcagaagcccggcaaggcccccaagctgctgatctactacaccagcaacctgcacagcggcgtgcccagcaggtttagcggcagcggcagcggcaccgacttcaccctgaccatcagctccctgcagcccgaggacttcgccacctactactgccagcagtacaggaagctgccctggaccttcggccagggcaccaagctggagatcaagaggttcgtgcccgtgttcctgcccgccaaacccacaaccactcccgcccccaggccccctacacccgctcccaccattgccagccagcccctcagcctgaggccagaggcctgtaggcccgctgccggaggcgctgtgcacaccaggggcctggacttcgcctgcgacatctacatctgggcacccctggctggcacctgcggcgtgctgctgctgagcctggtgatcaccctgtactgcaaccacaggaacaagaggggcaggaagaagctgctgtacatcttcaagcagcccttcatgaggcccgtgcagaccacccaggaggaggacggctgctcttgcaggttccccgaagaggaggagggcggctgcgagctgggaggagggggaagcggaggcggcggctcaggcggcggcggaagcggcggcggcggcagcgaccccagcagcatggacagcaggctgcagaggatccacgccgagatcaagaacagcctcaagatcgacaacctggacgtgaacaggtgcatcgaggccctggacgagctggccagcctgcaggtgaccatgcagcaggcccagaagcacaccgagatgatcaccaccctgaagaagatcaggaggttcaaggtcagccaggtgatcatggagaagagcaccatgctgtacaacaagttcaagaacatgttcctggtcggagagggagacagcgtcatcacccaggtcctgaacaagagcgaccccggcggcggcagcggcagcggagccaccaatttcagcctgctgaagcaggccggagacgtggaggagaatcccggccccttcgaaatggccctgcccgtgaccgccctgctcctgcccctggccctgctgctgcacgccgcaaggcccgactataaggaccacgacggcgactacaaggaccacgacatcgactacaaggacgacgacgacaaaaccaggggcctggacttcgcctgcgacatctacatctgggcccccctggccggcacttgcggcgtgctgctgctgagcctcgtgatcaccctgtactgcaaccacaggaacaagcggggcaggaagaagctcctgtacatcttcaagcagcccttcatgaggcccgtgcagacaacccaggaggaggacggctgcagctgcaggttcccagaggaagaggagggcggctgcgagctggggggcggcggcagcggaggcggaggcagcggcggcggcggcagcggcggcggcggcagcggaggcagctgcagccccggcatctggcagctggactgcacccacctggaggggaaggtgattctggtggccgtgcacgtggcaagcggctatatcgaggccgaggtgattcccgccgagaccggccaggagaccgcctattttctgctcaagctggccggccggtggcccgtcaaaaccgtgcacaccgacaacggcagcaacttcaccagcaccaccgtcaaggccgcctgttggtgggccggcatcaagcaggagttcggcatcccctataacccccagagccagggcgtgatcgagagcatgaataaggagctgaagaagatcatcggccaggtgagggaccaggccgagcacctgaagaccgccgtgcagatggccgtgttcatccacaacaagaagaggaaggggggcatcggcggctacagcgccggcgagaggatcgtggatatcatcgccaccgacatctcagatccctcaggcggcggcggcagcggcggcggcggcagcggcggcggcggcagcagggtgaaatttagcaggagcgccgacgcccccgcctaccagcagggccagaaccagctgtacaacgagctgaacctgggccgcagggaggaatacgacgtgctggacaaaaggaggggcagggacccagagatgggcggcaagcccaggaggaagaacccccaggagggcctgtacaacgagctgcagaaggacaagatggccgaggcctacagcgagatcgggatgaaaggcgaaaggaggagaggcaagggccacgacggcctgtaccagggcctgagcaccgccactaaggacacctacgacgccctgcacatgcaggccctgccccccagg

SEQ ID NO: 43 - DNA, 3009 bp, Artificial
  Construct 10 DNA sequence
atggctctccccgtgaccgccctgctgctgcccctggccctgctgctgcacgccgccaggccccaggtgcagctggtccagagcggcgccgaggtgaagaagcctggcagcagcgtgaaggtgagctgcaagggcagcggctacaccttcaccaactactggatgcactgggtgaggcaggcccccggccagggcctggagtggattggggccacctacaggggccacagcgacacctactacaaccagaagttcaagggcagggccaccctgaccgccgataccagcaccagcaccgcctacatggaactgagcagcctgaggtccgaggacaccgccgtgtactactgcaccagaggcgccatctacgacggctacgacgtgctcgacaactggggccagggcaccctggtgaccgtgtctagcggcggcggcggcagcggcggcggcggcagcggaggcggcggatcaggcggcggcggcagcgatatccagatgacccagagcccctcctctctgagcgccagcgtgggcgatagggtgaccatcacctgcagcgccagccaggacatcagcaactacctgaactggtaccagcagaagcccggcaaggcccccaagctgctgatctactacaccagcaacctgcacagcggcgtgcccagcaggtttagcggcagcggcagcggcaccgacttcaccctgaccatcagctccctgcagcccgaggacttcgccacctactactgccagcagtacaggaagctgccctggaccttcggccagggcaccaagctggagatcaagaggttcgtgcccgtgttcctgcccgccaaacccacaaccactcccgcccccaggccccctacacccgctcccaccattgccagccagcccctcagcctgaggccagaggcctgtaggcccgctgccggaggcgctgtgcacaccaggggcctggacttcgcctgcgacatctacatctgggcacccctggctggcacctgcggcgtgctgctgctgagcctggtgatcaccctgtactgcaaccacaggaacaagaggggcaggaagaagctgctgtacatcttcaagcagcccttcatgaggcccgtgcagaccacccaggaggaggacggctgctcttgcaggttccccgaagaggaggagggcggctgcgagctgggaggagggggaagcggaggcggcggctcaggcggcggcggaagcggcggcggcggcagcgaccccagcagcatggacagcaggctgcagaggatccacgccgagatcaagaacagcctcaagatcgacaacctggacgtgaacaggtgcatcgaggccctggacgagctggccagcctgcaggtgaccatgcagcaggcccagaagcacaccgagatgatcaccaccctgaagaagatcaggaggttcaaggtcagccaggtgatcatggagaagagcaccatgctgtacaacaagttcaagaacatgttcctggtcggagagggagacagcgtcatcacccaggtcctgaacaagagcctggccgagcagaggcagcacgaggaggccaacaagaccaaggaccagggaaagaagggacccaacaagaagctggagaaggagagcgaccccggcggcggcagcggcagcggagccaccaatttcagcctgctgaagcaggccggagacgtggaggagaatcccggccccttcgaaatggccctgcccgtgaccgccctgctcctgcccctggccctgctgctgcacgccgcaaggcccgactataaggaccacgacggcgactacaaggaccacgacatcgactacaaggacgacgacgacaaaaccaggggcctggacttcgcctgcgacatctacatctgggcccccctggccggcacttgcggcgtgctgctgctgagcctcgtgatcaccctgtactgcaaccacaggaacaagcggggcaggaagaagctcctgtacatcttcaagcagcccttcatgaggcccgtgcagacaacccaggaggaggacggctgcagctgcaggttcccagaggaagaggagggcggctgcgagctggggggcggcggcagcggaggcggaggcagcggcggcggcggcagcggcggcggcggcagcggaggcagctgcagccccggcatctggcagctggactgcacccacctggaggggaaggtgattctggtggccgtgcacgtggcaagcggctatatcgaggccgaggtgattcccgccgagaccggccaggagaccgcctattttctgctcaagctggccggccggtggcccgtcaaaaccgtgcacaccgacaacggcagcaacttcaccagcaccaccgtcaaggccgcctgttggtgggccggcatcaagcaggagttcggcatcccctataacccccagagccagggcgtgatcgagagcatgaataaggagctgaagaagatcatcggccaggtgagggaccaggccgagcacctgaagaccgccgtgcagatggccgtgttcatccacaacaagaagaggaaggggggcatcggcggctacagcgccggcgagaggatcgtggatatcatcgccaccgacatctcagatccctcaggcggcggcggcagcggcggcggcggcagcggcggcggcggcagcagggtgaaatttagcaggagcgccgacgcccccgcctaccagcagggccagaaccagctgtacaacgagctgaacctgggccgcagggaggaatacgacgtgctggacaaaaggaggggcagggacccagagatgggcggcaagcccaggaggaagaacccccaggagggcctgtacaacgagctgcagaaggacaagatggccgaggcctacagcgagatcgggatgaaaggcgaaaggaggagaggcaagggccacgacggcctgtaccagggcctgagcaccgccactaaggacacctacgacgccctgcacatgcaggccctgccccccagg

SEQ ID NO: 44 - DNA, 2886 bp, Artificial
  Construct 11 DNA sequence
atggctctccccgtgaccgccctgctgctgcccctggccctgctgctgcacgccgccaggccccaggtgcagctggtccagagcggcgccgaggtgaagaagcctggcagcagcgtgaaggtgagctgcaagggcagcggctacaccttcaccaactactggatgcactgggtgaggcaggcccccggccagggcctggagtggattggggccacctacaggggccacagcgacacctactacaaccagaagttcaagggcagggccaccctgaccgccgataccagcaccagcaccgcctacatggaactgagcagcctgaggtccgaggacaccgccgtgtactactgcaccagaggcgccatctacgacggctacgacgtgctcgacaactggggccagggcaccctggtgaccgtgtctagcggcggcggcggcagcggcggcggcggcagcggaggcggcggatcaggcggcggcggcagcgatatccagatgacccagagcccctcctctctgagcgccagcgtgggcgatagggtgaccatcacctgcagcgccagccaggacatcagcaactacctgaactggtaccagcagaagcccggcaaggcccccaagctgctgatctactacaccagcaacctgcacagcggcgtgcccagcaggtttagcggcagcggcagcggcaccgacttcaccctgaccatcagctccctgcagcccgaggacttcgccacctactactgccagcagtacaggaagctgccctggaccttcggccagggcaccaagctggagatcaagaggttcgtgcccgtgttcctgcccgccaaacccacaaccactcccgcccccaggccccctacacccgctcccaccattgccagccagcccctcagcctgaggccagaggcctgtaggcccgctgccggaggcgctgtgcacaccaggggcctggacttcgcctgcgacatctacatctgggcacccctggctggcacctgcggcgtgctgctgctgagcctggtgatcaccctgtactgcaaccacaggaacaagaggggcaggaagaagctgctgtacatcttcaagcagcccttcatgaggcccgtgcagaccacccaggaggaggacggctgctcttgcaggttccccgaagaggaggagggcggctgcgagctgggaggagggggaagcggaggcggcggctcaggcggcggcggaagcggcggcggcggcagcgaccccagcagcatggacagcaggctgcagaggatccacgccgagatcaagaacagcctcaagatcgacaacctggacgtgaacaggtgcatcgaggccctggacgagctggccagcctgcaggtgaccatgcagcaggcccagaagcacaccgagatgatcaccaccctgaagaagatcaggaggttcaaggtcagccaggtgatcatggagaagagcaccatgctgtacaacaagttcaagaacatgttccagaggggaagcgaccccggcggcggcagcggcagcggagccaccaatttcagcctgctgaagcaggccggagacgtggaggagaatcccggccccttcgaaatggccctgcccgtgaccgccctgctcctgcccctggccctgctgctgcacgccgcaaggcccgactataaggaccacgacggcgactacaaggaccacgacatcgactacaaggacgacgacgacaaaaccaggggcctggacttcgcctgcgacatctacatctgggcccccctggccggcacttgcggcgtgctgctgctgagcctcgtgatcaccctgtactgcaaccacaggaacaagcggggcaggaagaagctcctgtacatcttcaagcagcccttcatgaggcccgtgcagacaacccaggaggaggacggctgcagctgcaggttcccagaggaagaggagggcggctgcgagctggggggcggcggcagcggaggcggaggcagcggcggcggcggcagcggcggcggcggcagcggaggcagctgcagccccggcatctggcagctggactgcacccacctggaggggaaggtgattctggtggccgtgcacgtggcaagcggctatatcgaggccgaggtgattcccgccgagaccggccaggagaccgcctattttctgctcaagctggccggccggtggcccgtcaaaaccgtgcacaccgacaacggcagcaacttcaccagcaccaccgtcaaggccgcctgttggtgggccggcatcaagcaggagttcggcatcccctataacccccagagccagggcgtgatcgagagcatgaataaggagctgaagaagatcatcggccaggtgagggaccaggccgagcacctgaagaccgccgtgcagatggccgtgttcatccacaacaagaagaggaaggggggcatcggcggctacagcgccggcgagaggatcgtggatatcatcgccaccgacatctcagatccctcaggcggcggcggcagcggcggcggcggcagcggcggcggcggcagcagggtgaaatttagcaggagcgccgacgcccccgcctaccagcagggccagaaccagctgtacaacgagctgaacctgggccgcagggaggaatacgacgtgctggacaaaaggaggggcagggacccagagatgggcggcaagcccaggaggaagaacccccaggagggcctgtacaacgagctgcagaaggacaagatggccgaggcctacagcgagatcgggatgaaaggcgaaaggaggagaggcaagggccacgacggcctgtaccagggcctgagcaccgccactaaggacacctacgacgccctgcacatgcaggccctgccccccagg

SEQ ID NO: 45 - DNA, 2886 bp, Artificial
  Construct 12 DNA sequence
atggctctccccgtgaccgccctgctgctgcccctggccctgctgctgcacgccgccaggccccaggtgcagctggtccagagcggcgccgaggtgaagaagcctggcagcagcgtgaaggtgagctgcaagggcagcggctacaccttcaccaactactggatgcactgggtgaggcaggcccccggccagggcctggagtggattggggccacctacaggggccacagcgacacctactacaaccagaagttcaagggcagggccaccctgaccgccgataccagcaccagcaccgcctacatggaactgagcagcctgaggtccgaggacaccgccgtgtactactgcaccagaggcgccatctacgacggctacgacgtgctcgacaactggggccagggcaccctggtgaccgtgtctagcggcggcggcggcagcggcggcggcggcagcggaggcggcggatcaggcggcggcggcagcgatatccagatgacccagagcccctcctctctgagcgccagcgtgggcgatagggtgaccatcacctgcagcgccagccaggacatcagcaactacctgaactggtaccagcagaagcccggcaaggcccccaagctgctgatctactacaccagcaacctgcacagcggcgtgcccagcaggtttagcggcagcggcagcggcaccgacttcaccctgaccatcagctccctgcagcccgaggacttcgccacctactactgccagcagtacaggaagctgccctggaccttcggccagggcaccaagctggagatcaagaggttcgtgcccgtgttcctgcccgccaaacccacaaccactcccgcccccaggccccctacacccgctcccaccattgccagccagcccctcagcctgaggccagaggcctgtaggcccgctgccggaggcgctgtgcacaccaggggcctggacttcgcctgcgacatctacatctgggcacccctggctggcacctgcggcgtgctgctgctgagcctggtgatcaccctgtactgcaaccacaggaacaagaggggcaggaagaagctgctgtacatcttcaagcagcccttcatgaggcccgtgcagaccacccaggaggaggacggctgctcttgcaggttccccgaagaggaggagggcggctgcgagctgggaggagggggaagcggaggcggcggctcaggcggcggcggaagcggcggcggcggcagcgaccccagcagcatggacagcaggctgcagaggatccacgccgagatcaagaacagcctcaagatcgacaacctggacgtgaacaggtgcatcgaggccctggacgagctggccagcctgcaggtgaccatgcagcaggcccagaagcacaccgagatgatcaccaccctgaagaagatcaggaggttcaaggtcagccaggtgatcatggagaagagcaccatgctgtacaacaagttcaagaacatgttccagcagggaagcgaccccggcggcggcagcggcagcggagccaccaatttcagcctgctgaagcaggccggagacgtggaggagaatcccggccccttcgaaatggccctgcccgtgaccgccctgctcctgcccctggccctgctgctgcacgccgcaaggcccgactataaggaccacgacggcgactacaaggaccacgacatcgactacaaggacgacgacgacaaaaccaggggcctggacttcgcctgcgacatctacatctgggcccccctggccggcacttgcggcgtgctgctgctgagcctcgtgatcaccctgtactgcaaccacaggaacaagcggggcaggaagaagctcctgtacatcttcaagcagcccttcatgaggcccgtgcagacaacccaggaggaggacggctgcagctgcaggttcccagaggaagaggagggcggctgcgagctggggggcggcggcagcggaggcggaggcagcggcggcggcggcagcggcggcggcggcagcggaggcagctgcagccccggcatctggcagctggactgcacccacctggaggggaaggtgattctggtggccgtgcacgtggcaagcggctatatcgaggccgaggtgattcccgccgagaccggccaggagaccgcctattttctgctcaagctggccggccggtggcccgtcaaaaccgtgcacaccgacaacggcagcaacttcaccagcaccaccgtcaaggccgcctgttggtgggccggcatcaagcaggagttcggcatcccctataacccccagagccagggcgtgatcgagagcatgaataaggagctgaagaagatcatcggccaggtgagggaccaggccgagcacctgaagaccgccgtgcagatggccgtgttcatccacaacaagaagaggaaggggggcatcggcggctacagcgccggcgagaggatcgtggatatcatcgccaccgacatctcagatccctcaggcggcggcggcagcggcggcggcggcagcggcggcggcggcagcagggtgaaatttagcaggagcgccgacgcccccgcctaccagcagggccagaaccagctgtacaacgagctgaacctgggccgcagggaggaatacgacgtgctggacaaaaggaggggcagggacccagagatgggcggcaagcccaggaggaagaacccccaggagggcctgtacaacgagctgcagaaggacaagatggccgaggcctacagcgagatcgggatgaaaggcgaaaggaggagaggcaagggccacgacggcctgtaccagggcctgagcaccgccactaaggacacctacgacgccctgcacatgcaggccctgccccccagg

SEQ ID NO: 46 - DNA, 2973 bp, Artificial
  Construct 13 DNA sequence
atggctctccccgtgaccgccctgctgctgcccctggccctgctgctgcacgccgccaggccccaggtgcagctggtccagagcggcgccgaggtgaagaagcctggcagcagcgtgaaggtgagctgcaagggcagcggctacaccttcaccaactactggatgcactgggtgaggcaggcccccggccagggcctggagtggattggggccacctacaggggccacagcgacacctactacaaccagaagttcaagggcagggccaccctgaccgccgataccagcaccagcaccgcctacatggaactgagcagcctgaggtccgaggacaccgccgtgtactactgcaccagaggcgccatctacgacggctacgacgtgctcgacaactggggccagggcaccctggtgaccgtgtctagcggcggcggcggcagcggcggcggcggcagcggaggcggcggatcaggcggcggcggcagcgatatccagatgacccagagcccctcctctctgagcgccagcgtgggcgatagggtgaccatcacctgcagcgccagccaggacatcagcaactacctgaactggtaccagcagaagcccggcaaggcccccaagctgctgatctactacaccagcaacctgcacagcggcgtgcccagcaggtttagcggcagcggcagcggcaccgacttcaccctgaccatcagctccctgcagcccgaggacttcgccacctactactgccagcagtacaggaagctgccctggaccttcggccagggcaccaagctggagatcaagaggttcgtgcccgtgttcctgcccgccaaacccacaaccactcccgcccccaggccccctacacccgctcccaccattgccagccagcccctcagcctgaggccagaggcctgtaggcccgctgccggaggcgctgtgcacaccaggggcctggacttcgcctgcgacatctacatctgggcacccctggctggcacctgcggcgtgctgctgctgagcctggtgatcaccctgtactgcaaccacaggaacaagaggggcaggaagaagctgctgtacatcttcaagcagcccttcatgaggcccgtgcagaccacccaggaggaggacggctgctcttgcaggttccccgaagaggaggagggcggctgcgagctgggaggagggggaagcggaggcggcggctcaggcggcggcggaagcggcggcggcggcagcgaccccagcagcatggacagcaggctgcagaggatccacgccgagatcaagaacagcctcaagatcgacaacctggacgtgaacaggtgcatcgaggccctggacgagctggccagcctgcaggtgaccatgcagcaggcccagaagcacaccgagatgatcaccaccctgaagaagatcaggaggttcaaggtcagccaggtgatcatggagaagagcaccatgctgtacaacaagttcaagaacatgttcctggtcggagagggagacagcgtcatcacccaggtcctgaacaagagcctggccgagcagaggcagcacgaggaggccaacaagaccaaggaccagggaaagaagggacccaacaagaagctggagaaggagagcgaccccggcggcggcagcggcagcggagccaccaatttcagcctgctgaagcaggccggagacgtggaggagaatcccggccccttcgaaatggccctgcccgtgaccgccctgctcctgcccctggccctgctgctgcacgccgcaaggcccgagcagaagttgatctcagaggaggatcttaccaggggcctggacttcgcctgcgacatctacatctgggcccccctggccggcacttgcggcgtgctgctgctgagcctcgtgatcaccctgtactgcaaccacaggaacaagcggggcaggaagaagctcctgtacatcttcaagcagcccttcatgaggcccgtgcagacaacccaggaggaggacggctgcagctgcaggttcccagaggaagaggagggcggctgcgagctggggggcggcggcagcggaggcggaggcagcggcggcggcggcagcggcggcggcggcagcggaggcagctgcagccccggcatctggcagctggactgcacccacctggaggggaaggtgattctggtggccgtgcacgtggcaagcggctatatcgaggccgaggtgattcccgccgagaccggccaggagaccgcctattttctgctcaagctggccggccggtggcccgtcaaaaccgtgcacaccgacaacggcagcaacttcaccagcaccaccgtcaaggccgcctgttggtgggccggcatcaagcaggagttcggcatcccctataacccccagagccagggcgtgatcgagagcatgaataaggagctgaagaagatcatcggccaggtgagggaccaggccgagcacctgaagaccgccgtgcagatggccgtgttcatccacaacaagaagaggaaggggggcatcggcggctacagcgccggcgagaggatcgtggatatcatcgccaccgacatctcagatccctcaggcggcggcggcagcggcggcggcggcagcggcggcggcggcagcagggtgaaatttagcaggagcgccgacgcccccgcctaccagcagggccagaaccagctgtacaacgagctgaacctgggccgcagggaggaatacgacgtgctggacaaaaggaggggcagggacccagagatgggcggcaagcccaggaggaagaacccccaggagggcctgtacaacgagctgcagaaggacaagatggccgaggcctacagcgagatcgggatgaaaggcgaaaggaggagaggcaagggccacgacggcctgtaccagggcctgagcaccgccactaaggacacctacgacgccctgcacatgcaggccctgccccccagg

SEQ ID NO: 47 - protein, 83 aa, Homo sapiens
SMDSRLQRIHAEIKNSLKIDNLDVNRCIEALDELASLQVTMQQAQKHTEMITTLKKIRRFKVSQVIMEKSTMLYNKFKNMFLV

SEQ ID NO: 48 - protein, 96 aa, Homo sapiens
SMDSRLQRIHAEIKNSLKIDNLDVNRCIEALDELASLQVTMQQAQKHTEMITTLKKIRRFKVSQVIMEKSTMLYNKFKNMFLVGEGDSVITQVLNK

SEQ ID NO: 49 - protein, 125 aa, Homo sapiens
SMDSRLQRIHAEIKNSLKIDNLDVNRCIEALDELASLQVTMQQAQKHTEMITTLKKIRRFKVSQVIMEKSTMLYNKFKNMFLVGEGDSVITQVLNKSLAEQRQHEEANKTKDQGKKGPNKKLEKE

SEQ ID NO: 50 - protein, 83 aa, Artificial
  Leu428Gln and Val429Arg mutations of LEGF/p75 domain (Uniprot O75475 residues 347-430)
SMDSRLQRIHAEIKNSLKIDNLDVNRCIEALDELASLQVTMQQAQKHTEMITTLKKIRRFKVSQVIMEKSTMLYNKFKNMFQR

SEQ ID NO: 51 - protein, 83 aa, Artificial
  Leu428Gln and Val429Gln mutations of LEGF/p75 domain (Uniprot O75475 residues 347-430)
SMDSRLQRIHAEIKNSLKIDNLDVNRCIEALDELASLQVTMQQAQKHTEMITTLKKIRRFKVSQVIMEKSTMLYNKFKNMFQQ

SEQ ID NO: 52 - protein, 507 aa, Artificial
  Construct 14 amino acid sequence
MALPVTALLLPLALLLHAARPQVQLVQSGAEVKKPGSSVKVSCKGSGYTFTNYWMHWVRQAPGQGLEWIGATYRGHSDTYYNQKFKGRATLTADTSTSTAYMELSSLRSEDTAVYYCTRGAIYDGYDVLDNWGQGTLVTVSSGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - DNA, 1521 bp, Artificial
  Construct 14 DNA sequence
atggccctgcccgtgaccgccctcctgctgcccctggccctgctgctgcacgccgccaggccccaggtccagctggtgcagagcggggccgaggtgaagaagcccggcagctccgtgaaagtgagctgcaagggcagcggctacaccttcaccaactactggatgcactgggtgaggcaggcccccggacagggactggagtggatcggcgccacctacaggggccacagcgacacctactacaaccagaagttcaagggcagggccaccctgaccgccgacactagcaccagcaccgcctacatggaactgagctcactgcggagcgaggacaccgccgtgtactactgcaccaggggcgccatctacgacggctacgacgtgctggacaactggggccagggcaccctggtgacagtgagctctggcggcggcgggagcggcggcggcggaagcggcggcggaggaagcggcggcggcggaagcgatatccagatgacccagagccccagcagcctgagcgccagcgtgggcgacagggtgaccatcacctgcagcgcaagccaggacatcagcaactacctgaactggtaccagcagaagcccggcaaggcccctaagctgctgatctactacacctctaacctgcacagcggcgtgcccagcaggttctctggcagcggctccggcaccgacttcactctgaccatcagcagcctccagcccgaggacttcgccacctactactgccagcagtacaggaagctcccctggaccttcggccagggcaccaagctggagatcaagcgcttcgtgcccgtgttcctccccgcaaaacccaccaccactcccgcccccagaccccccactcccgccccaacaattgccagccagcccctgagcctgaggcccgaggcttgtaggcccgccgctggcggcgccgtccacaccaggggcctggacttcgcctgcgacatctatatctgggcccccctggccggaacctgcggcgtgctgctgctgagcctggtgatcaccctgtactgcaaccacaggaacaagaggggcaggaagaagctcctgtacatcttcaagcagcccttcatgaggcccgtgcagaccacccaggaggaggacggctgcagctgcaggttcccagaggaagaggagggcgggtgcgaactgagagtgaaatttagcaggagcgccgacgcccccgcctatcagcaaggccagaaccagctgtacaacgagctcaacctgggcaggagggaggagtacgacgtgctggacaagcggaggggcagagatcccgagatgggcggcaagcccaggaggaagaatccccaggagggcctgtacaacgagctgcagaaggacaagatggccgaggcctacagcgagatcggcatgaagggggagaggaggaggggcaagggccacgacggcctgtaccagggcctgagcaccgccaccaaggacacctacgacgccctgcacatgcaggccctgccccccagg